(19) United States
(12) Patent Application Publication
(10) Pub. No.: US 2010/0173323 A1
(43) Pub. Date: Jul. 8, 2010

(54) GLYCOSYLATION ENGINEERED ANTIBODY THERAPY

(75) Inventors: Scott Strome, Reisterstown, MD (US); Lai-Xi Wang, Ellicott City, MD (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: UNIVERSITY OF MARYLAND, BALTIMORE, Baltimore, MD (US)

(21) Appl. No.: 12/304,080

(22) PCT Filed: Jun. 9, 2007
(86) PCT No.: PCT/US07/70818

§ 371 Date: Dec. 9, 2008

Related U.S. Application Data
(60) Provisional application No. 60/812,322, filed on Jun. 9, 2006.
(60) Provisional application No. 60/897,966, filed on Jan. 29, 2007.

(51) Int. Cl. G01N 33/53; C12P 21/06; C07K 16/00; G01N 33/566

(52) U.S. Cl. 435/7.1; 435/68.1; 530/391.1; 436/501

(57) ABSTRACT

The instant invention is drawn to methods of generating a glycosylation-engineered antibody, and using the glycosylation-engineered antibody for treating a patient, particularly a cancer patient or a patient with an immune disease or disorder. The instant invention is also drawn to methods of generating a glycosylation-engineered antibody for use in the treatment of patients having a polymorphism that does not respond to conventional antibody therapy. The instant invention is also drawn to methods of improving the biological activity of an antibody by glycosylation engineering. The instant invention is also drawn to methods of modulating antibody-dependent cell-mediated cytoxicity (ADCC) using a glycosylation-engineered antibody.

### CROSS-REFERENCE TO RELATED APPLICATIONS

This application is a national stage entry under 35 U.S.C. §371 of international application number PCT/US2007/070818, filed Jun. 9, 2007, which claims the benefit of U.S. Provisional Application 60/812,322 filed 9 Jun. 2006 entitled “Fc Receptor Polymorphisms for Solid Tumors as Prognostic for Antibody-Mediated Therapy” and claims the benefit of U.S. Provisional Application 60/897,966 filed 29 Jan. 2007 entitled “Glycosylation-Engineered Antibody Therapy.” The contents of these priority documents are hereby incorporated by reference in their entireties.

This invention was made with U.S. government support under grant number GM073717 awarded by the National Institutes of Health. The U.S. government has certain rights in this invention.

### BACKGROUND OF INVENTION

Monoclonal antibodies (mAbs) are emerging as an important class of therapeutic agents for the treatment of human diseases such as cancer [1, 2]. Currently used mAbs for cancer treatment are of IgG type and are produced in mammalian cells (CHO cells or mouse NSO cell lines etc.). Once recognizing the antigen and binding to the targets such as tumor cells, mAbs can trigger various effector functions, including: 1) antibody-dependent cell-mediated cytotoxicity (ADCC); 2) complement-dependent cytotoxicity (CDC); and/or 3) signal transduction changes, e.g., inducing cell apoptosis.

It is known that appropriate glycosylation at the conserved glycosylation site (N297) of the Fc domain is essential for the efficient interactions between mAbs and Fc receptors (FcR) and for the FcR-mediated effector functions, including antibody-dependent cell-mediated cytotoxicity (ADCC) and complement-dependent cytotoxicity (CDC). It was demonstrated that removing the N-glycan severely impairs ADCC and CDC. On the other hand, different forms of glycosylation (i.e., glycosylation states) exert significantly different effects, some are beneficial, while others are detrimental. For example, de-fucosylated, glycosylated HERCEPTIN was shown to be at least 50-fold more active in the efficacy of Fc-gamma receptor IIIa (FcgRIIIa) mediated ADCC than those with alpha-1,6-linked fucose residues [2b]. Similar results were reported for Ritximab and other mABs [2c, 2d]. Unfortunately, recombinant mAbs are produced currently via genetic engineering, with the result that the antibody protein is present as a mixture of glycosylation states (also known as glycoforms of the mAb), in which the more active glycoform (e.g., de-fucosylated and/or bisecting GlcNAc-containing N-glycans) may be present only in minor amounts or as a component of 5 or more glycoforms. All currently marketed mAbs are available as a mixture of mAb glycoforms as a result of their genetic engineering origin. Furthermore, glycosylation state has an effect on antibody-based treatments by, for example, increasing or decreasing ADCC.

Another factor in the overall efficacy of ADCC is the polymorphic nature of Fc gamma receptors (FcgR's). For example, lymphoma patients with homozygous amino acid position 158 valine/valine (V/V) alleles of FcgRIIIa (CD16a) [2e] or with Fc gamma receptor IIa (FcgRIIa) amino acid position 131 histidine/histidine (H/H) alleles demonstrate a higher response rate to rituxmab treatment. The 158V allele of FcgRIIIa (CD16a) and the 131H allele Fc g RIIa (FcgRIIa, CD32) have a higher affinity to human IgG1 than does the phenylalanine (F) allele and arginine (R) allele, respectively, resulting in more effective ADCC [3]. After multivariate analysis, these two Fc gamma receptor (FcgR) polymorphisms independently predicted longer progression free survival [4]. In light of this, it is therapeutically advantageous to purify or make recombinant mAbs with a particular glycosylation state optimized for affinity to particular FcgR polymorphisms to enhance ADCC, CDC, etc.

A typical immunoglobulin G (IgG) antibody is composed of two light and two heavy chains that are associated with each other to form three major domains connected through a flexible hinge region: the two identical antigen-binding (Fab) regions and the constant (Fc) region. The IgG-Fc region is a homodimer in which the two C_{H}3 domains are paired through non-covalent interactions. The two C_{H}2 domains are not paired but each has a conserved N-glycosylation site at Asn-297. After the antibody's recognition and binding to a target cell, ADCC and other effector functions are triggered through the binding of the antibody's Fc region to ligands such as FcgR's (FcgRI, FcgRII, and FcgRIII) on effector cells and the C1q component of complement. Essential effector functions of antibodies are dependent on appropriate glycosylation of the antibody's Fc region [5,6]. The IgG-Fc N-glycan exists naturally as a bi-antennary complex having considerable heterogeneity. The different IgG-Fc glycosylation states have been shown to elicit significantly different effector functions. Jeffries et al. have demonstrated that the core structure (Man3GlcNAc2) of the N297-glycan, particularly the initial three residues (ManGlcNAc2), is essential to confer significant stability and effector activity of antibody IgG-Fc [7-9]. Structural studies suggested that the N-glycan might exert its effects mainly through stabilization of the Fc domain's conformation [8, 10, 11].

Several groups have reported that the presence of the beta-1,4-linked bisecting GlcNAc residue in the core N297-glycan could significantly enhance the antibody's ADCC activity [12-14]. Subsequent studies suggested that the lack of the alpha-1,6-linked fucose residue, rather than the presence of the bisecting GlcNAc, might play a greater role in enhancing the antibody's ADCC activity [15]. Moreover, others have reported, with various conclusions, that the terminal Gal residues may or may not positively influence the effector functions [16-19]. It is noted that these studies have involved heterogeneous glycosylation states of the human IgG expressed in mammalian cell lines (e.g., CHO cell lines), and isolation of human IgG having a particular glycosylation state from this mixture is extremely difficult. Small amounts of impurities of a highly active species dramatically interferes with the results and data interpretation. Therefore, due to varying reports, unambiguous correlation of the effect on biological activity as a consequence of a specific IgG-Fc N-glycan structure (i.e., glycosylation state) remains undetermined.

Cellular glycosylation engineering has emerged as an attractive approach to obtain human-like, homogeneous glycoproteins for structural studies and for biomedical applications [6, 14, 20-24]. For example, over-expression of the GnTIII gene (responsible for adding the bisecting GlcNAc to the N-glycan) in a recombinant CHO cell-line led to the production of mAbs with enhanced population of bisecting GlcNAc, which showed an increased ADCC activity (via the higher affinity binding of the mAb to FcgRIII) [13,14]. Expression of mAbs in a FucT-8 knock-out CHO cells (lack of the alpha-1,6-fucosyltransferase) led to non-fucosylated or low-fucose containing glycosylation states of mAbs that showed enhanced ADCC [25, 26]. More recently, Gerngross et al. engineered a yeast Pichia pastoris system to express human-like mAbs de novo, which yielded typical bi-antennary complex type N-glycan lacking the alpha-1,6-fucose moiety [6]. Cellular glycoengineering showed great potential to produce glycoproteins with enhanced populations of the desired glycosylation states. However, cellular glycoengineering approaches available result in the production of heterogeneous mAbs having various glycosylation states. In addition, dramatic genetic engineering of an expressing system may result in instability and low expression efficiency of the host system. Therefore, a long felt need remains in the art for methods of producing homogeneous recombinant mAbs having particular glycosylation states, and their use in treating a subject in need thereof.

Other and further objects, features, and advantages will be apparent from the following description of the embodiments of the invention, which are given for the purpose of disclosure.

### BRIEF SUMMARY OF INVENTION

In one embodiment, the instant invention is drawn to a method of generating a glycosylation-engineered antibody comprising detecting an Fc Receptors (FcR) polymorphism in a sample, wherein said polymorphism is associated with poor responsiveness to a monoclonal antibody (mAb); de-glycosylating an Fc region of the mAb; and linking the deglycosylated Fc region of the mAb with a sugar to produce a glycosylation-engineered antibody having increased biological activity as compared to a non-glycosylation-engineered mAb. The instant invention is further drawn to the method, wherein a mAb is an IgG antibody, and in certain embodiments, an IgG1 antibody. The instant invention is further drawn to the method, wherein linking the deglycosylated Fc region of the mAb with a sugar is carried out by a transglycosylation reaction, such as, for example, to produce a beta-1,4 linkage.

In certain embodiments, the deglycosylation step comprises removal of at least one fucose, N-glycan, mannose, or the like from the Fc region.

In another embodiment, the instant invention is drawn to a method of generating a glycosylation-engineered antibody comprising detecting an Fc Receptors (FcR) polymorphism in a sample, wherein said polymorphism is associated with poor responsiveness to a monoclonal antibody (mAb); defucosylating the mAb; cleaving the mAb of a heterogeneous N-glycan, wherein the N-glycan is a sugar attached at position N-297 of the mAb; and linking the defucosylated and cleaved mAb with a sugar to produce a glycosylation-engineered antibody having increased biological activity as compared to a non-glycosylation-engineered mAb. The instant invention is further drawn to the method, wherein a mAb is an IgG antibody, and in certain embodiments, an IgG1 antibody. The instant invention is further drawn to the method, wherein linking the defucosylated and cleaved mAb with a sugar is carried out by a transglycosylation reaction, such as, for example, to produce a beta-1,4 linkage.

In another embodiment, the instant invention is drawn to a method of generating a glycosylation-engineered antibody comprising detecting an FcR polymorphism in a sample, wherein said polymorphism is associated with poor responsiveness to a monoclonal antibody (mAb); de-glycosylating an Fc region of the mAb; and linking the deglycosylated Fc region of the mAb with a sugar to produce a substantially pure glycosylation-engineered antibody having increased biological activity as compared to a non-glycosylation-engineered mAb. The instant invention is further drawn to the method, wherein a mAb is an IgG antibody, and in certain embodiments, an IgG1 antibody. The instant invention is further drawn to the method, wherein linking the deglycosylated mAb with a sugar is carried out by a transglycosylation reaction, such as, for example, to produce a beta-1,4 linkage.

In another embodiment, the instant invention is drawn to a method of treating a cancer subject comprising, detecting an FcR polymorphism in a sample, wherein said polymorphism is associated with poor responsiveness to an antibody therapy; generating a glycosylation-engineered antibody, wherein the glycosylation-engineered antibody has an increased biological activity as compared to the antibody therapy; and administering to the cancer subject the glycosylation-engineered antibody.

In another embodiment, the instant invention is drawn to a method of treating a cancer subject comprising, detecting an FcR polymorphism in a sample, wherein said polymorphism is associated with poor responsiveness to an antibody therapy; determining a glycosylation-engineered antibody, wherein the glycosylation-engineered antibody has an increased biological activity compared to the antibody therapy; and administering to the cancer subject the glycosylation-engineered antibody.

In another embodiment, the instant invention is drawn to a method of treating a subject having an immune-related disease or disorder comprising, detecting an FcR polymorphism in a sample, wherein said polymorphism is associated with poor responsiveness to an antibody therapy; generating a glycosylation-engineered antibody, wherein the glycosylation-engineered antibody has an increased biological activity compared to the antibody therapy; and administering to the subject having an immune-related disease or disorder the glycosylation-engineered antibody.

In another embodiment, the instant invention is drawn to a method of treating a subject in need thereof, wherein said method comprises administering a glycosylation-engineered antibody wherein said antibody induces or inhibits a co-stimulatory molecule or pathway. The instant invention is further drawn to the method, wherein a subject in need thereof comprises a cancer subject or a subject having an immune-related disease or disorder. The instant invention is further drawn to the method, wherein a subject in need thereof has or does not have an FcR polymorphism. The instant invention is further drawn to the method, wherein a co-stimulatory molecule or pathway is induced or inhibited in a target cell or in another cell other than a target cell.

In another embodiment, the instant invention is drawn to a method of controlling toxicity comprising administering to a subject in need thereof a glycosylation-engineered antibody having a disassociation constant for an FcR, which modulates biological activity when compared to a non-glycosylation-engineered antibody.

The methods described herein may apply to an instance wherein a desired subject and/or target has or lacks an FcR polymorphism.

The instant invention is further drawn to the method, wherein modulated includes an increase or decrease in biological activity.

In another embodiment, the instant invention is drawn to a method of modulating antibody-dependent cell-mediated cytotoxicity (ADCC) comprising administering a glycosylation-engineered antibody.

The methods of the present invention encompass modulated ADCC, which means an increase or a decrease in biological activity of the starting (control) mAb. The instant invention is further drawn to the method, wherein the corresponding FcR is an effector receptor, such as an Fc-g receptor (FcgR).

In another embodiment, the instant invention is drawn to a method of treating a subject in need thereof using an antibody having a desired glycosylation state to determine the effect of said glycosylation state on biological activity.

In another embodiment, the instant invention is directed to an antibody and a composition comprising the same that is generated by a method described herein. The instant invention is further drawn to the method wherein, an antibody is a mAb, preferably an IgG antibody, and in certain embodiments IgG1 antibody. Non-exemplary antibodies contemplated include a therapeutic glycosylation-engineered antibody wherein the starting antibody includes, but is not limited to, cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101 (Aphton), volociximab (Biogen Idec and PDL BioPharm), Anti-CD80 mAb (Biogen Idec), Anti-CD23 mAb (Biogen Idel), CAT-3888 (Cambridge Antibody Technology), CDP-791 (Imclone), eraptuzumab (Immunomedics), MDX-010 (Medarex and BMS), MDX-060 (Medarex), MDX-070 (Medarex), matuzumab (Merck), CP-675,206 (Pfizer), CAL (Roche), SGN-30 (Seattle Genetics), zanolimumab (Serono and Genmab), adecatumumab (Sereno), oregovomab (United Therapeutics), nimotuzumab (YM Bioscience), ABT-874 (Abbott Laboratories), denosumab (Amgen), AM 108 (Amgen), AMG 714 (Amgen), fontolizumab (Biogen Idec and PDL BioPharm), daclizumab (Biogent Idec and PDL BioPharm), golimumab (Centocor and Schering-Plough), CNTO 1275 (Centocor), ocrelizumab (Genetech and Roche), HuMax-CD20 (Genmab), belimumab (HGS and GSK), epratuzumab (Immunomedics), MLN1202 (Millennium Pharmaceuticals), visilizumab (PDL BioPharm), tocilizumab (Roche), ocrerlizumab (Roche), certolizumab pegol (UCB, formerly Celltech), eculizumab (Alexion Pharmaceuticals), pexelizumab (Alexion Pharmaceuticals and Procter & Gamble), abciximab (Centocor), ranibizimumab (Genetech), mepolizumab (GSK), TNX-355 (Tanox), or MYO-029 (Wyeth).

Another embodiment is directed to a method of producing an antibody having a desired glycosylation state comprising the steps of a) removing one or more sugars, b) chemically synthesizing a sugar, and c) enzymatically attaching the chemically synthesized sugar to (i) the antibody or (ii) a sugar attached to the antibody.

Another embodiment is directed to the method of paragraph [0024], wherein the chemically synthesized sugar comprises an oxazoline ring.

Another embodiment is directed to the method of paragraphs [0024] or [0025], wherein the enzyme is an endoglycosidase and the enzymatic attachment comprises a transglycosylation.

Another embodiment is directed to the method of paragraphs [0024]-[0026], wherein the sugar removed is an asparagine linked sugar, the polypeptide retains an N-acetylglucosamine at the asparagine after step a) and the enzymatic attachment is to the N-acetylglucosamine.

Another embodiment is directed to the method of paragraphs [0024]-[0027], wherein the antibody is a monoclonal antibody and the method results in substantially pure monoclonal antibody.

Another embodiment is directed to the method of paragraphs [0024]-[0028], wherein the chemically synthesized sugar results in a non natural carbohydrate structure after step c).

Another embodiment is directed to the method of paragraphs [0024]-[0029], wherein the substantially pure monoclonal antibody comprises a glycosylation state capable of modulating a biological activity.

Another embodiment is directed to the method of paragraphs [0024]-[0030], wherein the biological activity is (i) a binding affinity for an Fcg Receptor or (ii) antibody-dependent cell-mediated cytotoxicity.

Another embodiment is directed to the method of paragraphs [0024]-[0031], wherein the monoclonal antibody comprises cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101, volociximab, Anti-CD80 mAb, Anti-CD23 mAb, CAT-3888, CDP-791, eraptuzumab, MDX-010, MDX-060, MDX-070, matuzumab, CP-675,206, CAL, SGN-30, zanolimumab, adecatumumab, oregovomab, nimotuzumab, ABT-874, denosumab, AM 108, AMG 714, fontolizumab, daclizumab, golimumab, CNTO 1275, ocrelizumab, HuMax-CD20, belimumab, epratuzumab, MLN1202, visilizumab, tocilizumab, ocrerlizumab, certolizumab pegol, eculizumab, pexelizumab, abciximab, ranibizimumab, mepolizumab, TNX-355, or MYO-029.

Another embodiment is directed to an antibody composition comprising antibodies having a substantially pure glycosylation state.

Another embodiment is directed to the antibody composition of paragraph [0033], wherein the glycosylation state comprises at least four sugars.

Another embodiment is directed to the antibody composition of paragraph [0033] or [0034], wherein the antibody is a monoclonal antibody.

Another embodiment is directed to the antibody composition of paragraph [0033]-[0035], wherein the monoclonal antibody comprises cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101, volociximab, Anti-CD80 mAb, Anti-CD23 mAb, CAT-3888, CDP-791, eraptuzumab, MDX-010, MDX-060, MDX-070, matuzumab, CP-675,206, CAL, SGN-30, zanolimumab, adecatumumab, oregovomab, nimotuzumab, ABT-874, denosumab, AM 108, AMG 714, fontolizumab, daclizumab, golimumab, CNTO 1275, ocrelizumab, HuMax-CD20, belimumab, epratuzumab, MLN1202, visilizumab, tocilizumab, ocrerlizumab, certolizumab pegol, eculizumab, pexelizumab, abciximab, ranibizimumab, mepolizumab, TNX-355, or MYO-029.

Another embodiment is directed to a method of evaluating a biological activity of a glycopolypeptide comprising the steps of a) producing a substantially pure population of glycopolypeptides having a selected glycosylation state, and b) measuring the biological activity of the glycopolypeptide.

Another embodiment is directed to the method of paragraph [0037], wherein the glycopolypeptide is an antibody and the biological activity is (i) a binding affinity for an Fcg Receptor or (ii) antibody-dependent cell-mediated cytotoxicity.

Another embodiment is directed to the method of paragraph [0038], wherein the antibody comprises a monoclonal antibody.

Another embodiment is directed to the method of paragraphs [0038]-[0039], wherein the biological activity is antibody-dependent cell-mediated cytotoxicity in vivo.

Another embodiment is directed to the method of paragraphs [0038]-[0040], wherein the monoclonal antibody comprises cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101, volociximab, Anti-CD80 mAb, Anti-CD23 mAb, CAT-3888, CDP-791, eraptuzumab, MDX-010, MDX-060, MDX-070, matuzumab, CP-675,206, CAL, SGN-30, zanolimumab, adecatumumab, oregovomab, nimotuzumab, ABT-874, denosumab, AM 108, AMG 714, fontolizumab, daclizumab, golimumab, CNTO 1275, ocrelizumab, HuMax-CD20, belimumab, epratuzumab, MLN1202, visilizumab, tocilizumab, ocrerlizumab, certolizumab pegol, eculizumab, pexelizumab, abciximab, ranibizimumab, mepolizumab, TNX-355, or MYO-029.

Another embodiment is directed to a method of improving the outcome of an antibody based therapy comprising the steps of a) determining for a subject an Fcg Receptor allele present in the subject, and b) treating the subject with a monoclonal antibody comprising a substantially pure glycosylation state selected for (i) increased binding affinity to the Fcg Receptor allele present in the subject or (ii) increased antibody-dependent cell-mediated cytotoxicity.

Another embodiment is directed to the method of paragraph [0042], wherein the Fcg Receptor allele is an FcgIIIa Receptor allele for amino acid 158 or an FcgIIa Receptor allele for amino acid 131.

Another embodiment is directed to a method of selecting the glycosylation state for a monoclonal antibody comprising the steps of a) determining a Fcg Receptor allele on an immune cell, and b) selecting a glycosylation state which modulates, relative to a source monoclonal antibody having a heterogeneous glycosylation state, i) Antibody Dependent Cell Cytotoxicity, ii) Complement Dependent Cytotoxicity, iii) an Fc g receptor binding affinity, or iv) a monoclonal antibody induced cell signaling event.

Another embodiment is directed to a method of creating a bioequivalent of a monoclonal antibody comprising the steps of a) determining a glycosylation state for a pre-existing monoclonal antibody, and b) using the method of paragraphs [0024]-[0027] to produce a monoclonal antibody having substantially the same glycosylation state as the pre-existing monoclonal antibody.

In another embodiment, the instant invention is drawn to a method of modulating complement-dependent cytotoxicity (CDC) comprising administering a glycosylation-engineered antibody.

Another embodiment is directed to a method of creating a generic bioequivalent of a marketed MAb by producing an antibody having the desired glycosylation states comprising the steps of a) removing one or more sugars, b) chemically synthesizing sugars present in the marketed MAb, c) for each sugar enzymatically attaching the chemically synthesized sugars to (i) the antibody or (ii) a sugar attached to the antibody, and d) combining the MAb glycoforms in proportions substantially similar to the glycoform ratios present in the marketed MAb resulting in an antibody glycoform composition substantially matching the glycoform composition of a marketed antibody.

Another embodiment is directed to improving the efficacy, decreasing the toxicity, and/or decreasing the dose of a marketed MAb or a MAb that has been in clinical development by identifying a preferred MAb glycoform using a method of producing an antibody having a substantially pure glycosylation state comprising the steps of a) removing one or more sugars from the identified MAb, b) chemically synthesizing a preferred sugar present in the MAb, and c) enzymatically attaching the chemically synthesized sugar to (i) the antibody or (ii) a sugar attached to the antibody.

Another embodiment is directed to a method of selecting for clinical development a glycoform of a mAb for use in a population having a Fc g receptor allele comprising the steps of a) testing a glycoform of a mAb for biological activity against the Fcg Receptor alleles present in the population, and b) selecting for clinical development the mAb glycoform capable of (i) increased binding affinity to the Fcg Receptor allele present in the population or (ii) increased antibody-dependent cell-mediated cytotoxicity.

Another embodiment is directed to the method of paragraph [0049], wherein the Fcg Receptor allele is an FcgIIIa Receptor allele for amino acid 158 or an FcgIIa Receptor allele for amino acid 131.

Another embodiment is directed to a method of creating a substantially pure glycoform of a pre-existing monoclonal antibody having a heterogeneous glycosylation state comprising the steps of using the method of claims 1-4 to create two or more of the glycoforms present in the pre-existing monoclonal antibody, testing the two or more glycoforms for a biological activity or a toxicity to determine a preferred glycoform of the pre-existing monoclonal antibody having a higher biological activity or a lower toxicity, and using the method of paragraphs [0024]-[0027] to produce a monoclonal antibody glycoform having a substantially pure preferred glycosylation state identified in step b) as having a higher biological activity or a lower toxicity.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF DRAWINGS

FIG. 1 illustrates the interaction of Natural Killer (NK) cells with a tumor cell.

FIG. 2 describes an example of a glycosylation state for an antibody.

FIG. 3 describes restriction enzyme analysis of FcgRIII allelic forms from genomic DNA. Prior to restriction digestion, 40 mL of crude PCR product was cleaned with a phenol extraction followed by one phenol/isoamyl-chloroform extraction prior to ethanol precipitation. For Rsa I single digestion, 15 mL of cleaned PCR product was digested with 15 units of Rsa I overnight at 37° C. with 1× incubation buffer at final volume of 20 mL. For double digestion, 25 mL of cleaned PCR product was digested overnight with 20 units of Rsa I in a 30 mL final volume with 1× incubation buffer at 37° C., followed by the addition of 50 units of Eco130 I restriction enzyme with 1× incubation buffer, with incubation overnight at 37° C. All products were analyzed by electrophoresis on a 3% (w/w) agarose gel in TAE buffer. DNA from #1 demostrates FcgRIIIa F/F, #2 heterozygous F/V, and #3 homozygous V/V.

FIG. 4 describes restriction enzyme analysis of FcgRII allelic forms from genomic DNA. DNA was purified from 3 different individuals and after PCR, the products were digested with BstUI enzyme. The products were separated on an agarose gel and stained with ethidium bromide. The three possible genotypes were identified.

FIG. 5 outlines a glycosylation-engineering process applied to an IgG or IgG-Fc domain by a combined cellular and chemoenzymatic approach.

FIG. 6 shows an example synthesis of a substantially pure oligosaccharide oxazoline.

FIG. 7 shows an example glyco-transferase reaction to yield a peptide population having a substantially pure oligosaccharide content.

FIG. 8 shows an example glyco-transferase reaction to yield Ribonuclease B enzyme population having a substantially pure glycosylation state composed of the core N-linked pentasaccharide Man3G1cNAc2.

FIG. 9 shows an oligosaccharide synthesis scheme yielding a novel non-natural carbohydrate structure.

FIG. 10 shows freshly isolated NK cells incubated with HNSCC cell lines (Tu 167, Tu 159 or O12SCC). A. Untreated B. Treated with 10 ug/mL Cetuximab. Assessments were performed following 16 h incubation with ⁵¹Cr Assay and performed in triplicate. K562 cell line was used as positive control for each experiment, data not shown. NK purity was all greater than 90%.

FIG. 11A shows SDS-PAGE of recombinant yeast IgG₁-Fc domain protein. Lane 1 is the product having the starting yeast N-glycan. Lane 2 shows End-A deglycosylated IgG₁-Fc domain protein. Lane 3 shows the deglycosylated protein in lane 2 after chemoenzymatic transglycosylation with a synthetic hexasaccharide oxazoline. 11B shows SDS-PAGE of recombinant yeast IgG₁-Fc domain protein. Lane 1 is the product having the starting yeast N-glycan. Lane 2 shows the transglycosylated protein after chemoenzymatic transglycosylation with a synthetic hexasaccharide oxazoline. Lanes 3-4 and 5-6 show PNGase F deglycosylation of the starting yeast product from lane 1 and the transglycosylated IgG₁-Fc domain protein from lane 2, respectively.

### DETAILED DESCRIPTION OF INVENTION

As used in the specification herein, “a” or “an” may mean one or more. As used herein in the claim(s), when used in conjunction with the word “comprising”, the words “a” or “an” may mean one or more than one. As used herein “another” may mean at least a second or more.

As used herein, a “sample” refers typically to any type of material of biological origin including, but not limited to, a cell, fluid, tissue, or organ isolated from a subject, including, for example, blood, plasma, serum, fecal matter, urine, semen, bone marrow, bile, spinal fluid, lymph fluid, samples of the skin, external secretions of the skin, respiratory, intestinal, and genitourinary tracts, tears, saliva, milk, blood cells, organs, or biopsies.

As used herein, “biological activity” refers to pharmacodynamic and pharmacokinetic properties including, for example, molecular affinity or resultant biochemical or physiological effect, receptor affinity or resultant biochemical or physiological effect, non-receptor affinity or biochemical or physiological effect, efficacy, bioavailability, absorption, distribution, metabolism, or elimination.

As used herein, “sugar” refers to an oxidized or unoxidized carbohydrate-containing molecule, including, but not limited to, a monosaccharide, disaccharide, trisaccharide, oligosaccharide, or polysaccharide, including, for example, N-acetylglucosamine, mannose, galactose, N-acetylneuraminic acid (sialic acid), glucose, fructose, fucose, sorbose, rhamnose, mannoheptulose, N-acetylgalactosamine, dihydroxyacetone, xylose, xylulose, arabinose, glyceraldehyde, sucrose, lactose, maltose, trehalose, cellobiose, oligosaccharide oxazolines, a non-natural variant or analog of any of the foregoing, or any combination thereof of the L- or D-isomer. Sugar further refers to, such molecules produced naturally, recombinantly, synthetically, and/or semi-synthetically.

As used herein, “poor responsiveness” refers to a decrease in response rate, a decrease initial response rate, a decrease in survival rate, or a decrease in “biological activity”, as defined above, when compared to the majority of the population.

As used herein, “antibody-dependent cell-mediated cytotoxicity” (ADCC) refers to an immune response in which antibodies, by coating target cells, makes them vulnerable to attack by immune cells.

As used herein, “modulates” refers to an increase or decrease in biological activity, as defined above, when comparing to a gylcosylation-engineered antibody to a non-glycosylation-engineered antibody (starting antibody, control, or other equivalent terms).

As used herein, “cancer” refers to, a pathophysiological state whereby a cell is characterized by dysregulated and proliferative cellular growth and the ability to induce said growth, either by direct growth into adjacent tissue through invasion or by growth at distal sites through metastatsis in both, adults or children, and both acute or chronic, including, but not limited to, carcinomas and sarcomas, such as, acute lymphoblastic leukemia, acute myeloid leukemia, adrenocortical cancer, AIDS-related cancers, AIDS-related lymphoma, anal cancer, astrocytoma (cerebellar or cerebral), basal cell carcinoma, bile duct cancer, bladder cancer, bone cancer, brain stem glioma, brain tumor (e.g., ependymoma, medulloblastoma, supratentorial primitive neuroectodermal, visual pathway and hypothalamic glioma), cerebral astrocytoma/malignant glioma, breast cancer, bronchial adenomas/carcinoids, Burkitt's lymphoma, carcinoid tumor (e.g., gastrointestinal), carcinoma of unknown primary site, central nervous system lymphoma, cervical cancer, chronic lymphocytic leukemia, chronic myelogenous leukemia, chronic myeloproliferative disorders, colon cancer, colorectal cancer, cutaneous T-Cell lymphoma, endometrial cancer, ependymoma, esophageal cancer, Ewing's Family of tumors, extrahepatic bile duct cancer, eye cancer (e.g., intraocular melanoma, retinoblastoma, gallbladder cancer, gastric cancer, gastrointestinal carcinoid tumor, gastrointestinal stromal tumor (GIST), germ cell tumor (e.g., extracranial, extragonadal, ovarian), gestational trophoblastic tumor, glioma, hairy cell leukemia, head and neck cancer, squamous cell head and neck cancer, hepatocellular cancer, Hodgkin's lymphoma, hypopharyngeal cancer, islet cell carcinoma (e.g., endocrine pancreas), Kaposi's sarcoma, laryngeal cancer, leukemia, lip and oral cavity cancer, liver cancer, lung cancer (e.g., non-small cell), lymphoma, macroglobulinemia, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myeloma, nasal cavity and paranasal sinus cancer, nasopharyngeal cancer, neuroblastoma, non-Hodgkin's lymphoma, oral cancer, oral cavity cancer, osteosarcoma, oropharyngeal cancer, ovarian cancer (e.g., ovarian epithelial cancer, germ cell tumor), ovarian low malignant potential tumor, pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, prostate cancer, rectal cancer, retinoblastoma, rhabdomyosarcoma, salivary gland cancer, soft tissue sarcoma, uterine sarcoma, Sézary syndrome, skin cancer (e.g., non-melanoma or melanoma), small intestine cancer, supratentorial primitive neuroectodermal tumors, T-Cell Lymphoma, testicular cancer, throat cancer, thymoma and thymic carcinoma, thyroid cancer, transitional cell cancer of the renal pelvis and ureter, trophoblastic tumor (e.g. gestational), unusual cancers of childhood and adulthood, urethral cancer, endometrial uterine cancer, uterine sarcoma, vaginal cancer, vulvar cancer, Waldenström's macroglobulinemia, Wilms' Tumor, and women's cancers.

As used herein, “immune-related disease or disorder” refers to a disease or disorder wherein the immune system is enhanced or suppressed or in which a component of the immune system causes, mediates, or otherwise contributes to morbidity or morality. Also included are diseases in which stimulation or intervention of the immune response has an ameliorative effect on progression of the disease or disorder. Included within this term are immune-mediated inflammatory diseases, non-immune-mediated inflammatory diseases, infectious diseases, immunodeficiency diseases, cancer, etc., including, for example, systemic lupus erythematosis, amyotrophic lateral sclerosis, Parkinson's disease, Alzheimer's disease, rheumatoid arthritis, juvenile chronic arthritis, spondyloarthropathies, systemic sclerosis (e.g., scleroderma), idiopathic inflammatory myopathies (e.g., dermatomyositis, polymyositis), Sjogren's syndrome, sarcoidosis, autoimmune hemolytic anemia (e.g., immune pancytopenia, paroxysmal nocturnal hemoglobinuria), autoimmune thrombocytopenia (e.g., idiopathic thrombocytopenic purpura, immune-mediated thrombocytopenia, thrombotic thrombocytopenic purpura), thyroiditis (e.g., Grave's disease, Hashimoto's thyroiditis, juvenile lymphocytic thyroiditis, atrophic thyroiditis), diabetes mellitus, immune-mediated renal disease (e.g., glomerulonephritis, tubulointerstitial nephritis), demyelinating diseases of the central and peripheral nervous systems (e.g., multiple sclerosis), idiopathic demyelinating polyneuropathy or Guillain-Barre syndrome, multiple myositis, mixed connective tissue disease, hyperthyroidism, myasthenia gravis, autoimmune hepatopathy, autoimmune nephropathy, vasculitidies (e.g. Kawasaki's disease or temporal arterities), autoimmune hematopathy, idiopathic interstitial pneumonia, hypersensitivity pneumonitis, autoimmune dermatosis, autoimmune cardiopathy, cardiomyositis, autoimmune infertility, Behcet's disease, chronic inflammatory demyelinating polyneuropathy, hepatobiliary diseases (e.g., infectious hepatitis and other non-hepatotropic viruses), autoimmune chronic active hepatitis, primary biliary cirrhosis, granulomatous hepatitis, and sclerosing cholangitis, inflammatory bowel disease (e.g., ulcerative colitis: Crohn's disease), gluten-sensitive enteropathy, Whipple's disease, autoimmune or immune-mediated skin diseases including bullous skin diseases, vitiligo, erythema multiforme and contact dermatitis, psoriasis, sexually transmitted diseases, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease, viral diseases (e.g., AIDS (HIV infection), hepatitis A, B, C, D, and E, herpes), bacterial infections, fungal infections, protozoal infections and parasitic infections.

As used herein, with respect to antibodies, “substantially pure” means separated from those contaminants that accompany it in its natural state or those contaminants generated or used in the process of obtaining the antibody. This term further includes the desired product having a single glycosylation state, whether or not this state includes glycosylation at a single site or multiple sites. Typically, the antibody is substantially pure when it constitutes at least 60%, by weight, of the antibody in the preparation. For example, the antibody in the preparation is at least about 75%, in certain embodiments at least about 80%, in certain embodiments at about 85%, in certain embodiments at least about 90%, in certain embodiments at least about 95%, and most preferably at least about 99%, by weight, of the desired antibody. A substantially pure antibody includes a naturally, recombinantly, or synthetically produced antibody.

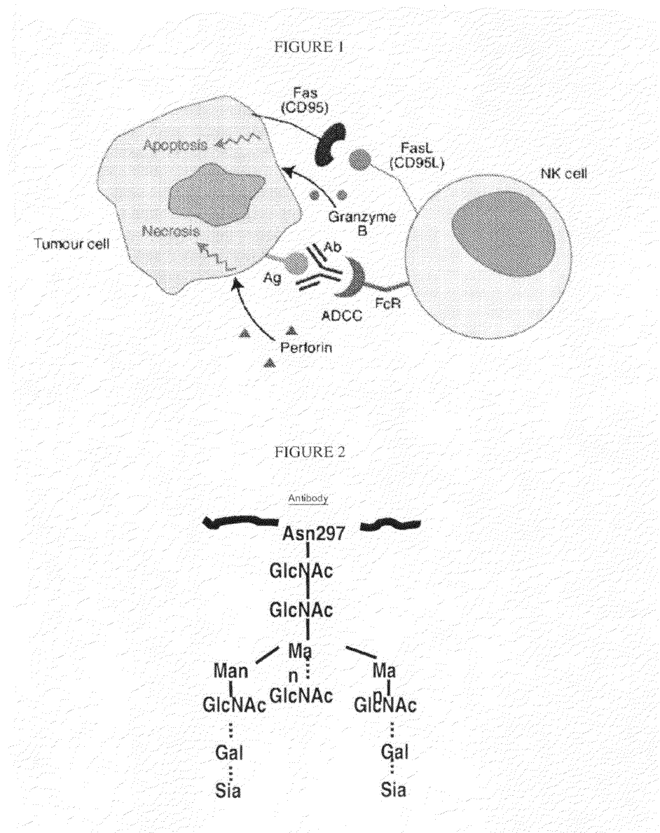

As used herein, “glycosylation state” refers to an antibody having a specific or desired glycosylation pattern. A “glycoform” is an antibody comprising a particular glycosylation state. Such glycosylation patterns include, for example, attaching one or more sugars at position N-297 of a mAb, wherein said sugars are produced naturally, recombinantly, synthetically, or semi-synthetically. By way of example, a mAb having a glycosylation state comprises an IgG₁ linked at position N-297 to at least one N-glycan and lacking an alpha-1,6-fucose is provided in FIG. 2.

As used herein, “antibody” refers to immune system-related proteins called immunoglobulins and their separately functional fragments. Each antibody consists of four polypeptides two heavy chains and two light chains joined to form a “Y” shaped molecule. Treating an antibody with a protease can cleave the protein to produce Fab or fragment antigen binding that include the variable ends of an antibody and/or the constant region fragment Fc. The constant region determines the mechanism used to destroy antigen (e.g. ADCC). Antibodies are divided into five major classes, IgM, IgG, IgA, IgD, and IgE, based on their constant region structure and immune function. These classes include subclasses such as IgG₁₋₄. An antibody may be polyclonal or monoclonal.

As used herein, “polypeptide” refers to a molecule comprising two or more amino acids covalently linked together. A “glycopolypeptide” refers to a polypeptide further comprising at least one sugar covalently linked to the polypeptide.

The term “treating” and “treatment” as used herein refers to administering to a subject a therapeutically effective amount of an antibody so that the subject has an improvement in a disease. The improvement is any improvement or remediation of the symptoms. The improvement is an observable or measurable improvement. Thus, one of skill in the art realizes that a treatment may improve the disease condition, but may not be a complete cure for the disease. Specifically, improvements in patients with cancer may include tumor stabilization, tumor shrinkage, increased time to progression, increased survival or improvements in the quality of life. Improvements in patients with autoimmune disease may include improvement in laboratory values of inflammation, improvements in blood counts, improvements in rash, or improvements in the quality of life.

The term “therapeutically effective amount” as used herein refers to an amount that results in an improvement or remediation of the symptoms of the disease or condition.

The term “subject” as used herein, is taken to mean any mammalian subject to which an antibody composition is administered according to the methods described herein. In a specific embodiment, the methods of the present invention are employed to treat a human subject. Another embodiment includes treating a human subject suffering from cancer.

NK Cell FcgR Polymorphisms

Antigen presenting cells (APC) such as NK cells play an integral role in antibody dependent cellular cytotoxicity (ADCC). NK cells possess cell surface receptors, FcgR's that bind IgG, which facilitates cross-linking with adjacent FcgR's and activation of the NK cell, leading to ADCC [26b]. The affinity of binding to an FcgR with resultant activation and cytotoxic effect is influenced by receptor polymorphisms. For example, lymphoma patients with homozygous valine/valine (V/V) alleles of FcgRIIIa (CD16a) at amino acid 158 or with FcgRIIa histidine/histidine alleles at amino acid 131 demonstrated a higher response rate to rituxmab treatment. The FcgRIIIa (CD16a) of V allele and FcgRIIa (CD32) of H allele have a higher affinity to human IgG1 than does the phenylalanine (F) allele and arginine (R) allele, respectively, resulting in more effective ADCC.[3] After multivariate analysis, these two FcgR polymorphisms independently predicted longer progression free survival.[4]

The correlation between which FcgRIIIa allele NK cells express and ADCC has recently been confirmed in vitro. HNSCC cell lines TU 167, TU159 and O12SCC were used in this study. ADCC assays were performed using HNSCC cells as target cells, and purified NK cells as effector cells. Target cells were incubated with 150 μCi Cr-51 (Amersham, Piscataway, N.J.) at 37° C. for 1 hour, mixing well every 15 minutes, and then washed twice with media. Cells were subsequently incubated with 10 ug/mL of Cetuximab, 10 ug/mL of human IgG1 isotype, or media alone for another 30 minutes at 37° C., and then washed twice to remove unbound antibodies. Effector and target cells were plated in 96 well plates and incubated overnight. Cell lysis supernatant was collected and mixed with Optiphase Supermix scintillation fluid (Perkin Elmer, Boston Mass.) and counted in a MicroBeta 1450 scintillation counter (Wallac, Turku Findland). The results were expressed as the percentage of specific lysis: [(Experimental cpm-spontaneous cpm)×100]/(maximum cpm-spontaneous cpm).

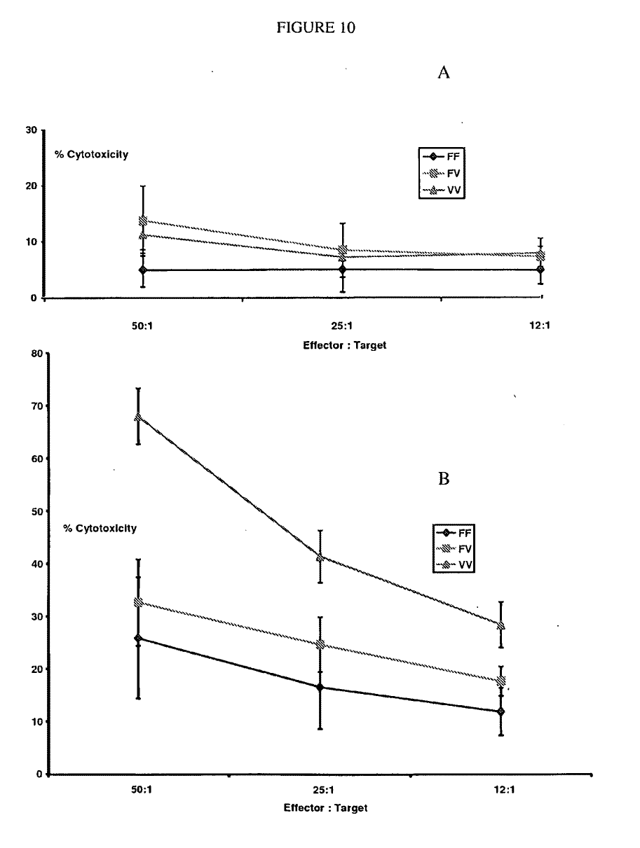

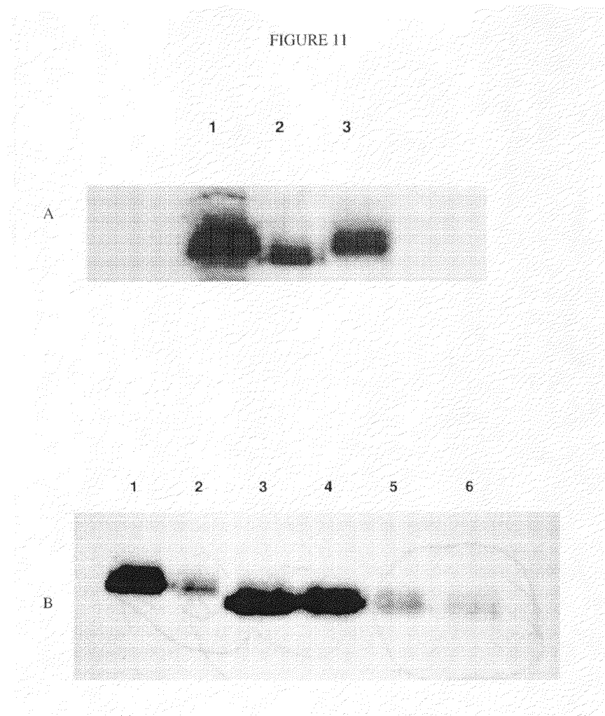

FIG. 10A demonstrates untreated fresh NK cells in the absence of antibody with each FcgRIIIa polymorphism incubated with the HNSCC cell lines. Their killing ability measured with ⁵¹Cr ranges from 0-26%, with a median ranging from 5-15%. FIG. 10B is a representation of the mean killing of Cetuximab-treated-HNSCC cell lines that were incubated with NK cells. In comparison to untreated HNSCC cell lines, Cetuximab-treated HNSCC cell lines demonstrate a significantly higher killing activity. Moreover, FcgRIIIa polymorphism V/V mediates killing superior to V/F and F/F when incubated with 10 μg/mL Cetuximab of HNSCC cell lines. In general at a 50:1 effector to target ratio, all cell lines show low cytotoxic activity when incubated with FcgRIIIa F/F NK donor, moderate cytotoxic activity when incubated with FcgRIIIa F/V NK donor, and high cytotoxic activity when incubated with FcgRIIIa V/V NK donor.

These data provide in vitro evidence that CD16a polymorphisms are associated with differential antibody dependent cytotoxicity levels against HNSCC. Presumably, it is the differential binding affinity of each NK FcgRIIIa polymorphic genotype to the Fc portion of Cetuximab that underlies the difference in NK-mediated cytotoxicity. Knowing which polymorphism that a patient has at the beginning of therapy may be predictive of the overall tumor response and clinical outcome for monoclonal antibody. Ultimately, optimizing the binding of NK FcgRIIIa alleles to the Fc portion of bound mAb will improve ADCC for each polymorphism. Carbohydrate structures imbuing mAbs with improved affinities for FcgRIIIa (CD16a) 158F alleles will be particularly important for enhancing treatment outcome in carriers of these alleles.

### Example 1

### Detection of FcgRIIIa Receptor (CD16a) and FcgRIIa (CD32) Allelic Polymorphisms

In order to determine the ability of glycosylation-engineered mAbs to induce ADCC in a patient with diverse genotypes or to determine the responsiveness of non-glycoengineered mAbs, PCR based strategies, for example, are used to characterize allelic variants for position 131 of FcgRIIa and position 158 of FcgRIIIa. First, genomic DNA was isolated from human tumor cells lines, human saliva, human PBMC or paraffin embedded tissue and was used as a template for PCR amplification.

A. Detection of the FcgIIIa Receptor (CD16a) Allelic Polymorphism Using PCR Amplification and Restriction Enzyme Digestion.

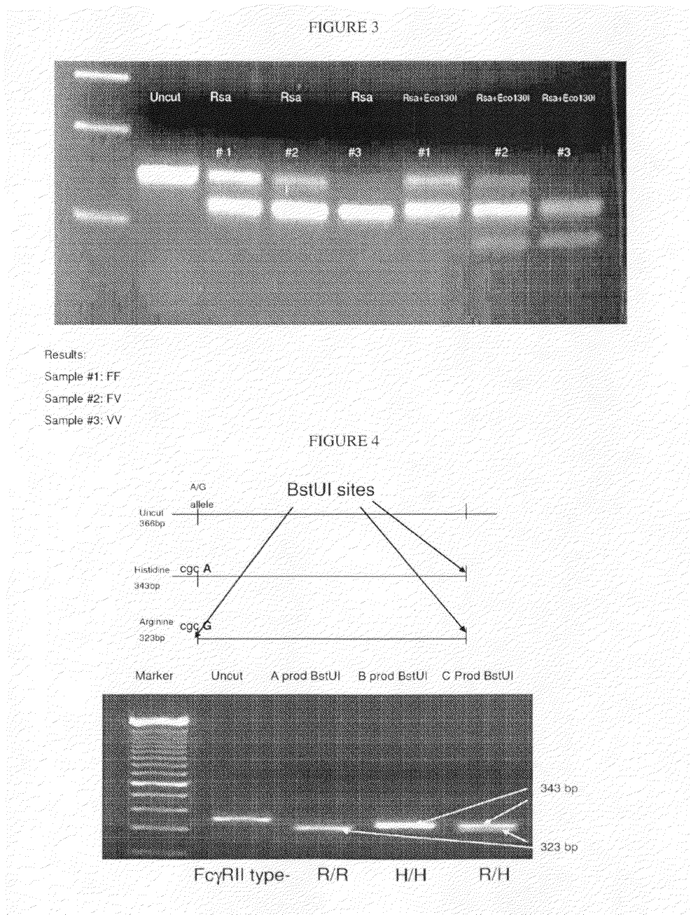

Primer design is based on sequences available in GenBank (accession no. X52645 for FcgRIIIa, Nieto et al, 2000). This procedure uses primers that introduced a novel RsaI site into one end of all amplified products and a second primer that created a novel StyI (or Eco130 I) site in one of the two FcgRIIIa alleles. The sense primer (5′-ATAAGGTCACATATTTACAGAATGGCCAAG-3′) (SEQ ID NO: 1) and the antisense primer (5′-CAGTCTCTGAAGACACATTTTTACTCCGTA-3′) (SEQ ID NO: 2) amplify a 147 by fragment containing the polymorphic site. Mismatch shown in bold for the sense primer creates restriction site (StyI) in either allele of FcgRIIIa genes, but not the FcgRIIIb gene. The mismatch in the antisense primer creates a restriction site (RsaI) in FcgRIIIa, only for the V allele, and in the FcgRIIIb gene. In the case of FcgRIII both the FcgRIIIa and b genes are both amplified because of sequence similarity. To differentiate alleles for FcgRIIIa, for example, two restriction enzyme digestions are preformed, one with RsaI and a second digestion with StyI or Eco130 I (StyI and Eco130 I both recognize the same sequence). Table 1 shows restriction enzyme digest patterns for the two alleles of FcgRIIIa and FIG. 3 illustrates actual restriction enzyme digests.

TABLE 1

| Genotype | RSAI | RSAI + Eco130I |
|---|---|---|
| FcgRIIIa V/V | 119 bp only | 119 bp + 91 bp |
| FcgRIIIa V/F | 147 bp + 119 bp | 119 bp + 91 bp |
| FcgRIIIa F/F | 147 bp + 119 bp | only 119 bp |

B. Detection of Restriction Polymorphisms of the FcgRIIa Receptor Using PCR Amplification and Restriction Enzyme Digestion.

Primer design was based on McKenzie et al., 1996, which uses a sense primer (5′-GGAAAATCCCAGAAATTCTCGC-3′) (SEQ ID NO: 3) and the antisense (5′-CAACAGCCTGACTACCTATTACGCGGG-3′) (SEQ ID NO: 4) to amplify a 366 by fragment containing the polymorphic site. One nucleotide substitution in the sense primer, shown in bold, introduces a BstUI cut site into the PCR product when the next nucleotide is G, but not when the next nucleotide is A. A second BstU I is put into the antisense primer to control for digestion. Amplification with both primers will introduce a restriction enzyme site in the C terminus for both products of both alleles. But only one allele will contain a second restriction, the arginine (R) site. When the PCR products are digested with restriction enzyme BstUI the R alleles will be digested twice, yielding a short product (323 bp) while the histidine containing alleles will only cut once producing a 343 by band. FIG. 4 illustrates the three possible types that will be observed. Products A and B are the digestion products of homozygous individuals arginine (R/R) and histidine (H/H) respectively. Product C shows what a heterozygous individual (R/H) demonstrates. An internal control of BstUI was designed at the end of the reverse primer to ensure successful BstUI digestion.

C. Polymorphism Detection and Correlation to Antibody-Based Therapy.

Following the detection of a polymorphism as described in the immediately preceding sections A. and B. above, correlation to antibody-based therapy responsiveness follows. In the alternative, responsiveness to antibody-based therapy may be determined followed by the detection of a polymorphism. In light of a particular polymorphism, the clinician or other appropriate professional staff determines the responsiveness of glycosylation-engineered or non-glycosylation-engineered antibody therapy by establishing whether or not the patient carrying a particular polymorphism responds to therapy using a glycosylation-engineered or non-glycosylation-engineered antibody. By correlating a polymorphism with responsiveness to a glycosylation-engineered or non-glycosylation-engineered antibody therapy, a prediction regarding responsiveness to a glycosylation-engineered or non-glycosylation-engineered antibody can be made.

### Prophetic Example 2

### Homogenous Preparation of Antibodies

To obtain a homogeneous preparation of mAbs with a particular glycosylation state, a combined high-yield cellular expression with in vitro glycosylation engineering using a chemoenzymatic transglycosylation system is utilized [27-30]. Combined with the power of chemical synthesis of oligosaccharide oxazoline substrates for the endo-enzymes, this approach allows for the preparation of an array of defined glycosylation states (natural or unnatural) of mAbs or their IgG-Fc domain, which, in turn, allows for a systematic analysis of the structure-activity relationships of IgG glycosylation and ADCC activity. Following the pioneering work of Jeffries et al., use of the hingeless human IgG-Fc, the delta-h-Fc (aa 231-447) as a model system, in which the hinge region of Fc was deleted, is also used [7, 31]. Using this truncated Fc form rather than a whole human antibody IgG or IgG-Fc as a model system greatly simplifies the synthesis as well as the subsequent structure-function relationship studies. Results from hingless IgG-Fc experiments may be confirmed by expression and transglycosylation of whole IgG. In addition, the Fc portion of IgG may be expressed and modified by the same transglycosylation process to produce novel Fc fragments with homogenous, synthesized carbohydrate contents.

At least two expression systems can be used for expressing the hingeless IgG-Fc. The instant invention is not limited by the expression systems described herein. One expression system is the CHO-K1 cell system that was previously used to overproduce human delta-h-Fc glycoprotein [7, 31]. The plasmid encoding the delta-h-Fc gene (aa231-447) is constructed in exactly the same way as reported, using the commercially available plasmid pg1 L243H as a source of the C_{H} g1 gene [7, 31]. The system produces a delta-h-Fc glycoprotein with a heterogeneous N-glycan. Another expression system is a high-yield yeast mutant expression system, which produces the IgG-Fc glycoprotein with a high-mannose type oligosaccharide attached. After overproduction and subsequent purification, the resulting glycoprotein delta-h-Fc is treated with a mixture of Endo-F2 or Endo-M and a fucosidase (to remove the heterogeneous sugar chains expressed from the CHO-cell line), or treated with Endo-H or Endo-A (to remove the high-mannose type oligosaccharides produced from the yeast system). This removes all the heterogeneous N297-glycans, while leaving only the inner most GlcNAc attached at the glycosylation site. Subsequently, the resulting GlcNAc-containing IgG-Fc serve as the acceptor substrate for transglycosylation to add back various homogeneous oligosaccharides from sugar oxazolines under the catalysis of a suitable endo-enzyme or its mutants [30]. Using various synthetic sugar oxazolines as the donor substrates, the ENGase-catalyzed transglycosylation provides various glycosylation states of delta-h-Fc, Fc domain proteins and mAbs with defined oligosaccharide structure. These include the N-glycan core structures, those with fucose and those with bisecting GlcNAc structure. It also includes selected modified structures that may further contribute to ADCC activity. The general approach is depicted in the FIG. 5. In addition to the method described above, this approach applies to whole IgG antibody preparations. The disclosure also is not restricted in scope or breadth and includes, for example, methods, peptides, and antibodies as described in U.S. Pat. No. 7,138,371 (DeFrees et al.) [32].

### Example 3

### Example Design and Synthesis of Carbohydrate Oxazolines

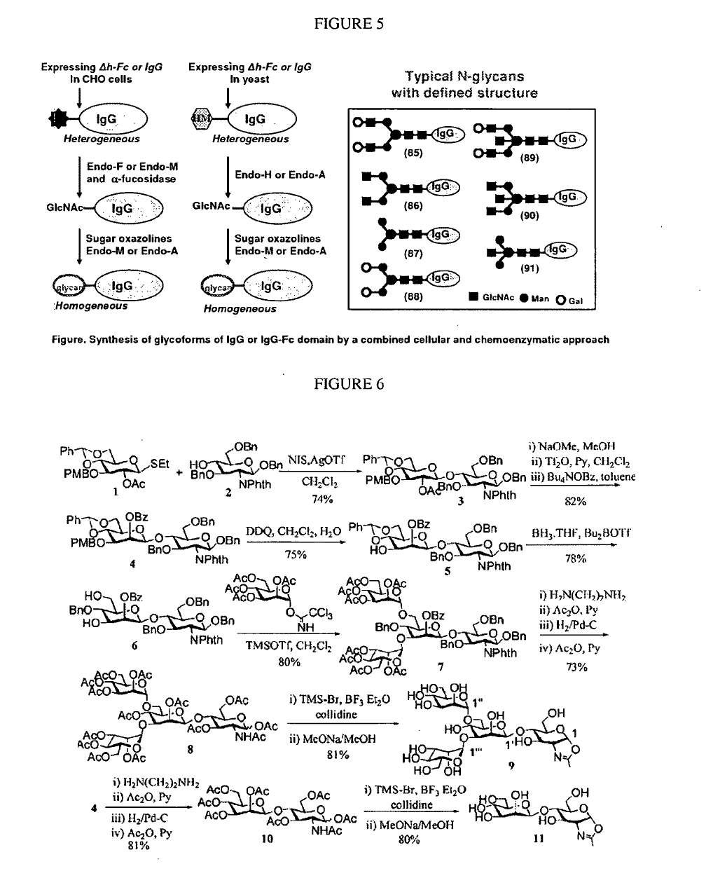

ENGases are a class of endoglycosidases that hydrolyze the beta-1,4-glycosidic bond in the core N,N′-diacetylchitobiose moiety of N-glycoproteins to release the N-glycans. However, some ENGases, such as Endo-A from Arthrobacter protophormiae and Endo-M from Mucor hiemalis, possess transglycosylation activity and are able to transfer the releasing N-glycan to a GlcNAc-peptide acceptor to form a new glycopolypeptide. Endo-A and Endo-M can transfer a large intact oligosaccharide to a GlcNAc-peptide acceptor in a single step to form a new glycopolypeptide, thus allowing a highly convergent glycopolypeptide synthesis without the need of protecting groups. The chemoenzymatic method suffers with a low transglycosylation yield (generally 5-20%), product hydrolysis, and the limitations of using only natural N-glycans as the donor substrates. To solve these problems, we used synthetic oligosaccharide oxazolines, the mimics of the presumed oxazolinium ion intermediate formed in a retaining mechanism, as donor substrates for glycopolypeptide synthesis. We synthesized the di- and tetrasaccharide oxazolines corresponding to the core of N-glycans. To test whether oligosaccharide oxazolines would be kinetically more favorable substrates for an efficient N-glycopolypeptide synthesis than natural N-glycans. The basic synthetic scheme is shown in FIG. 6 [33].

### Example 4

### Transglycosylation of Oligosaccharide Oxazoline Substrates onto an HIV gp41 Fragment

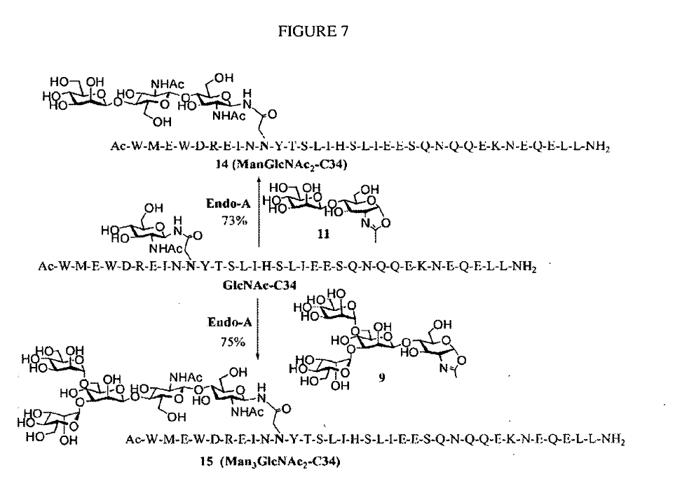

We next tested the Endo-A-catalyzed transglycosylation of the di- and tetrasaccharide oxazolines with the large acceptor, GlcNAc-C34 (FIG. 7). It was found that the oligosaccharides could also be effectively transferred to the large GlcNAc-C34 by Endo-A to form the glycopeptides 14 (73%) and 15 (75%), respectively. The glycopeptides were characterized by ESI-MS and NMR analysis. Further structural characterization of glycopolypeptide 15 was performed by Pronase digestion that yielded a single Asn-linked oligosaccharide, which was identical to the authentic Asn-linked core pentasaccharide Man3GlcNAc2Asn by 1H NMR, ESIMS, and Dionex HPAEC analysis. It was also observed that while the Man-beta1,4-GlcNAc-oxazoline and Man3GlcNAc-oxazoline acted as an efficient substrate for transglycosylation, the resulting glycopolypeptide ManGlcNAc2-C34 (14) was resistant to Endo-A hydrolysis, and the glycopolypeptide Man3GlcNAc2-C34 (15) was hydrolyzed only slowly by Endo-A. These results show that oligosaccharide oxazolines are more active substrates than the ground state N-glycopeptides, thus being kinetically favorable for product accumulation.

### Example 5

### Synthesis of a Normatural Hexasaccharide (Gal2Man3GlcNAc) Oxazoline

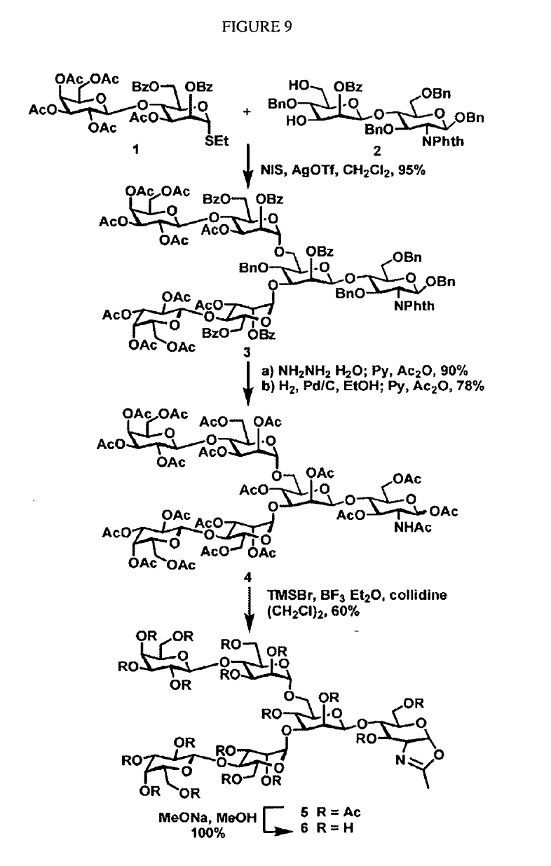

We designed and synthesized a nornnatural hexasaccharide (Gal2Man3GlcNAc) oxazoline, which has two galactose residues beta-1,4-linked to the terminal mannose residues in the Man3-G1cNAc core. This hexasaccharide derivative is a mimic of a bi-antennary complex type N-glycan without the interlinked GlcNAc moieties (FIG. 9). A model reaction was carried out with a small GlcNAc-peptide, Ac-Asn(G1cNAc)-Ile-Thr as the acceptor. The enzymatic reaction was monitored by reverse phase HPLC. The glycosylation of the acceptor with the hexasaccharide oxazoline by Endo-A was essentially complete within 30 minutes to form the glycopolypeptide having a substantially pure glycosylation state with a 98% yield.

### Example 6

### Transglycosylation of Oligosaccharide Oxazoline Substrates Onto RNAse B

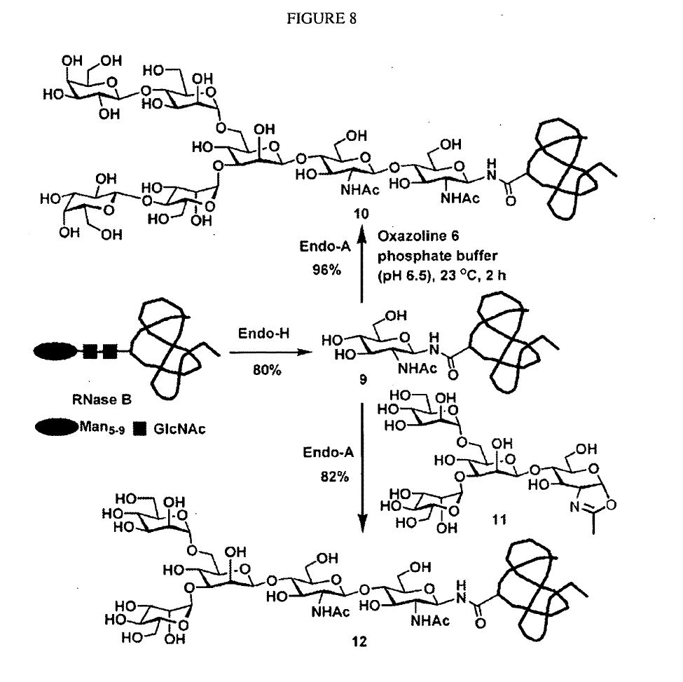

To examine the feasibility of the chemoenzymatic method for glycoprotein synthesis and remodeling, bovine ribonuclease B was chosen as a model system. Treatment of ribonuclease B with Endo-H removed the N-glycans, leaving only the innermost N-acetylglucosamine(G1cNAc) at the Asn-34 site and producing substantially pure GlcNAc-RB. It was found that when the hexasaccharide oxazoline 6 (FIG. 8) and GlcNAc-RB (molar ratio, 2:1) were incubated in a phosphate buffer (pH 6.5) at 23° C. in the presence of Endo-A, the GlcNAc-RB was glycosylated to give the trans-glycosylation product 10. The transformation was essentially quantitative after 2 h reaction and the substantially pure glycoprotein product was isolated in 96% yield. Similarly, Endo-A catalyzed reaction of GlcNAc-RB with the tetrasaccharide oxazoline 11 gave substantially pure glycoprotein 12 carrying the core N-linked pentasaccharide Man3GlcNAc2 with an 82% yield. The efficient attachment of the core N-linked pentasaccharide (Man3GlcNAc2) to a protein will provide a key starting structure for a quick assembly of a variety of glycosylation states via sequential glycosylations of the core with various glycosyltransferases.

### Example 7

### Transglycosylation of a Hexasaccharide onto Recombinant Fc Domain

The coding sequence for the human IgG1-Fc domain was amplified by PCR and cloned into a yeast expression vector pYES2/CT (INVITROGEN). The resulting IgG1-Fc-pYES2/CT was transformed into an OCH-1 mutant of Saccharomyces cerevisiae [44] and expressed. SDS-PAGE confirmed that the purified IgG1-Fc is glycosylated and PNGase F treatment revealed the quantitative removal of the N-glycan. The native IgG1-Fc appeared as a 35 KDa band under reduced condition, corresponding to the monomeric form, but appeared as a 70 KDa band under native condition, indicating that the purified IgG1-Fc is associated as a dimer as is found in the native IgG1 structure. The expressed glycoprotein was purified and used as a transglycosylation target protein.

To examine the feasibility of chemoenzymatic remodeling of an antibody glycoform, we used the IgG1-Fc portion produced in yeast as described above. Our preliminary studies revealed that Endo-A can successfully remove the heterogeneous high-mannose type N-glycan from yeast expressed IgG1-Fc to produce a GlcNAc-IgG₁-Fc, which appeared as a band of about 33 KDa (FIGS. 5 and 11A-B). The hexasaccharide oxazoline (Gal2Man3GlcNAc-oxazoline) was used as a model sugar oxazoline for these antibody transglycosylation reactions. This sugar oxazoline was previously demonstrated as an excellent substrate of Endo-A for transglycosylation remodeling of ribonuclease B [30]. When the GlcNAc-IgG1-Fc was incubated with the hexasaccharide oxazoline in the presence of Endo-A, a newly glycosylated IgG1-Fc was formed, which appeared on SDS-PAGE at a size similar to the original recombinant glycosylated IgG1-Fc (FIGS. 11A & B). This result indicated that the transglycosylation is equally efficient for the IgG1-Fc as the ribonuclease B model system. To confirm that the transferred oligosaccharide was attached to the GlcNAc in the protein, we treated the newly formed glycosylated IgG1-Fc with PNGase F, which can remove the N-glycan only when the glycan is attached in the GlcNAc-Asn linkage. As shown in FIG. 11B, treatment of original IgG1-Fc and the remodeled glycosylated IgG1-Fc resulted in deglycosylated IgG1-Fc with identical sizes as judged by SDS-PAGE. These data indicate that the transglycosylation hexasaccharide was attached to the GlcNAc-Asn formed by Endo-A as expected. Further detailed N-glycan analysis are carried out with MALDI-TOF and ESI mass spec.

Epidermal Growth Factor Receptor (EGFR) and mAb C225 (Cetuximab).

EGFR is a member of the erbB family of receptor tyrosine kinases. When ligand binds, dimerisation and oligomerisation ensue and activation of the cytoplasmic protein tyrosine kinase occurs. Downstream and second messenger signaling follows, promoting cell proliferation and survival/antiapoptotisis via the activation of transcription factors and upregulation of cyclin D1 [33b].

Over expression is seen in a variety of solid tumors and is associated with a higher stage, increased lymph node metastasis, shorter relapse-free survival and overall survival [33c]. Ang et al. demonstrated that over expression in SCCHN is associated with decrease survival and an independent predictor of locoregional relapse [33d]. Targeted therapy directed against EGFR with chimeric mAb C225 (Cetuximab) for advanced SCCHN in combination with standard chemoradiation protocols has emerged as an important therapy. Cetuximab is an IgG1 monoclonal antibody against the ligand-binding domain of EGFR and prevents activation of the tyrosine kinase. Phase II and III trials have demonstrated improved clinical outcomes using Cetuximab [33e, 33c].

### Prophetic Example 8

### Transglycosylation of Oligosaccharide Oxazoline Substrates onto mAb C225 and its delta-h-Fc Counterpart

The human-mouse chimeric anti-EGF receptor mAB C225 with heterogeneous carbohydrate attachments to ASN297 or a delta-h-Fc version of mAB C225 are treated with Endo-H leaving the innermost N-acetylglucosamine(G1cNAc) on ASN297. The Endo-H treated mAB C225 is combined with the core N-linked pentasaccharide (Man3GlcNAc2) 11 and Endo-H or a similar glycolytic enzyme with transglycosylation activity. Routine purification techniques yield substantially pure, homogenously glycosylated mAb C225. The core N-linked pentasaccharide is further modified by additional glycosylations using standard glycotransferase reactions to derive a variety of substantially pure mAb C225 glycosylation states. See, e.g., [40].

### Prophetic Example 9

### Effector Functions of Glycosylation-Engineered delta-h-Fc mAb C225 Antibodies

The effector functions of various glycosylation states of delta-h-Fc mAB C225 are first examined by receptor binding assays. Several FcgR's are tested, including FcgRIIb (inhibitory receptor), FcgRIIIa 158V, and FcgRIIIa 158F (receptor polymorphisms). The binding assays follow the reported procedures [6]. The binding studies reveal a set of particular glycosylation states that demonstrate high-affinity binding to FcgRIIIa (both V and F variants) while possessing low affinity for FcgRIIb. Particular glycosylation states are identified that show improved binding properties.

The effector functions of the various glycosylation states of delta-h-Fc are also examined for their ability to interact with human FcgRI by a competitive inhibition assay, following the reported procedure [7, 8, 31]. Briefly, U937 leukocyte cells are stimulated with gamma-IF/V to induce differentiation and expression of human FcgRI. Target JY cells are sensitized with a humanized IgG1. After incubation with serial concentrations of particular glycosylation states of delta-h-Fc C225 and lucigenin, the sensitized JY cells are mixed with the U937 effector cells and the superoxide production is measured as indicated by the change in chemiluminecence. The inhibitory activity is compared for different glycosylation states of the delta-h-Fc C225. This study reveals how individual sugar residues in the N-glycan contribute to effector functions. Particularly, this study unambiguously clarifies the role of the bisecting GlcNAc residue in enhancing effector functions. In addition to the structure-relationship activity studies described above, this approach also applies to whole IgG antibody expression and glycosylation remodeling to produce those glycosylation states with high-affinity binding to effector cells, such as, the NK cells that stimulate ADCC activity. Taken together, these studies provide important information on the functional role of the N-glycans on IgG-Fc and form the basis for enhancing effector functions of therapeutic monoclonal antibodies through specific glycosylation states.

Analysis of Structure-Function Relationship of Glycosylation-Engineered mAbs.

While in vitro models of ADCC are useful for initial characterization of the function of glycosylation-engineered mAbs, in vivo models provide further data to support clinical translation. To specifically evaluate the utility of glycosylation-engineered forms of C225, or other therapeutic antibodies, to induce ADCC, a compound xenograft SCID mouse model, depleted of endogenous murine NK, is used for adoptively transferring NK cells bearing defined FcgR polymorphisms [34, 35]. Using this system allows for the evaluation of glycosylation-engineered antibodies to enduce ADCC [36, 37, 38]. Preferably the NK cells are from individuals homozygous for V/V or F/F at amino acid 158 of FcgRIIIa or H/H or R/R at amino acid 131 of FcgRIIa.

Different tumor cell lines are used to evaluate glyco-engineered C225 mAbs (native structure or hingless) having substantially pure glycosylation states. M24met is a melanoma cell line known to be responsive to C225 antibody treatment in this model system. This cell line expresses a mutant form of EGFR which binds both murine and chimeric 225 mAb without tyrosine kinase phosphorylation and subsequent EGFR signaling. Additional melanoma cell lines expressing no EGFR are identified by FACS analysis of available melanoma cell lines. An EGFR −/− cell is stabily transfected with a non-functional EGFR mutant which is expressed on the cell surface. Mice inoculated with wild type EGFR positive melanoma cell lines such as A431 and M21 are used to compare CHO cell line produced C225 with glyco-engineered forms of C225 which show ADCC with M24met and/or the stabily transfected melanoma cell line.

### Prophetic Example 10

### Growth of EGFR Mutant Human Tumor Cell lines in vivo

M24met and/or a human SCCHN cell line transfected with nonfunctional, expressed EGFR (e.g. a kinase activity mutant) are used to establish growth curves in SCID/SCID mice. Specifically, three days prior to tumor inoculation, animals are depleted of endogenous NK cells by tail vein injection of anti-asialo 1.1. A total of 6 animals (2 animals/group) are intradermally injected with 1×10⁵, 1×10⁶, or, 1×10⁷, cells in 0.1 ml of PBS. Tumor growth will be measured QOD and animals are sacrificed when the tumor reaches approximately 10% of body weight, when the tumor becomes ulcerated, when the animal is unable to access food or water, or when the animal is deemed by the investigators to be in a premorbid condition. At the time of sacrifice, lungs, liver and spleen are evaluated for the presence of metastatic disease. These studies define the parameters for tumor inoculation and growth into SCID mice.

### Prophetic Example 11

### Survival of Human NK Cells Following adoptive transfer into SCID mice

We purify CD56 positive cells from buffy coat blood using variomacs beads. In order to remove NKT cells, CD3 positive cells are depleted from this population. Three days prior to human NK transfer, mice are depleted of endogenous NK cells by IV injection of anti-asialo 1.1. On the day of transfer, NK cells are stained with CFSE and then 1×106, 1×107 or 5×107 cells are adoptively transferred in 0.5 cc of PBS via tail vein or intraperitoneal injection. One animal/group is sacrificed at weekly intervals and their peripheral blood, bone marrow and spleens are analyzed for the presence and proliferation of CFSE positive cells. In order to ensure efficacy of endogenous NK depletion, these same organ systems are evaluated for the presence of murine NK. These studies define the parameters for NK adoptive transfer into SCID mice.

### Prophetic Example 12

### In Vivo Evaluation of Glyco-Engineered C225 mAB

On Day 0, anti-asialo 1.1 antibody is injected to deplete endogenous murine NK cells. On Day 3, the melanoma tumor cell line is injected and tumors allowed to form on the basis of the results from Prophetic Example 10. On Day 6, human NK cells are adoptively transferred on the basis of the results from Prophetic Example 11. NK cells adoptively transferred may be selected to cover all combinations of CD16a and CD32 polymorphisms to identify the optimal glycosylation structures for specific receptor alleles. On days 7, 14 and 21, C225 mAB or a glyco-engineered C225 mAB with a substantially pure glycosylation state is injected based on the protocols in [39]. Treatment groups are illustrated in Table 2 (Glyco C225 is a glyco-engineered C225 mAb or a hingless equivalent).

TABLE 2

| | | NK | | |
|---|---|---|---|---|
| GROUP | # Animals | transfer | mAb | Purpose |
| 1 | 5 | None | None | Tumor growth control |
| 2 | 5 | None | C225 | C225 Control |
| 3 | 5 | None | Glyco C225 | Glyco C225 Control |
| 4 | 5 | Yes | None | Natural antitumor |
| | | | | activity of NK |
| 5 | 5 | Yes | C225 | ADCC activity of C225 |
| 6 | 5 | Yes | Glyco C225 | ADCC activity of Glyco |
| | | | | C225 |
| 7 | 5 | Yes | FcgIIIa | Control mAB which |
| | | | receptor | blocks CD16a and |
| | | | (CD16a) | inhibits ADCC activity |

### Prophetic Example 13

### Comparative In Vivo Evaluation of Glyco-Engineered C225 mAb to the Parent C225 mAb with Heterogeneous Glycosylation

Following the results of Prophetic Example 13, C225 mAbs with substantially pure glycosylation states are compared in vivo to the precursor C225 mAb. C225 with substantially pure glycosylation states is more effective at inhibiting tumor growth and/or reducing metastasis.

The glycosylation states that improve C225 mAb efficacy will do so by increasing the mAb's ability to induce ADCC. Thus, the identified carbohydrate structures will be suitable for improving the efficacy of any mAb which induces ADCC, including, but not limited to, cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101 (Aphton), volociximab (Biogen Idec and PDL BioPharm), Anti-CD80 mAb (Biogen Idec), Anti-CD23 mAb (Biogen Idel), CAT-3888 (Cambridge Antibody Technology), CDP-791 (Imclone), eraptuzumab (Immunomedics), MDX-010 (Medarex and BMS), MDX-060 (Medarex), MDX-070 (Medarex), matuzumab (Merck), CP-675,206 (Pfizer), CAL (Roche), SGN-30 (Seattle Genetics), zanolimumab (Serono and Genmab), adecatumumab (Sereno), oregovomab (United Therapeutics), nimotuzumab (YM Bioscience), ABT-874 (Abbott Laboratories), denosumab (Amgen), AM 108 (Amgen), AMG 714 (Amgen), fontolizumab (Biogen Idec and PDL BioPharm), daclizumab (Biogent Idec and PDL BioPharm), golimumab (Centocor and Schering-Plough), CNTO 1275 (Centocor), ocrelizumab (Genetech and Roche), HuMax-CD20 (Genmab), belimumab (HGS and GSK), epratuzumab (Immunomedics), MLN1202 (Millennium Pharmaceuticals), visilizumab (PDL BioPharm), tocilizumab (Roche), ocrerlizumab (Roche), certolizumab pegol (UCB, formerly Celltech), eculizumab (Alexion Pharmaceuticals), pexelizumab (Alexion Pharmaceuticals and Procter & Gamble), abciximab (Centocor), ranibizimumab (Genetech), mepolizumab (GSK), TNX-355 (Tanox), or MYO-029 (Wyeth).

Exemplary Medical Applications of Glyco-Engineered C225 (Cetuximab).

Racial Disparity for SCCHN.

As discussed above, particular alleles of FcgRIIIa and FcgRIIa correlate with a reduced efficacy of mAb induced ADCC. These genetic variations are likely represented in different racial and ethnic groups with differing frequencies. There is increased recognition of the public health impact of cancer disparities among ethnic groups, particularly African Americans. Tumor Registries were constructed, in part, so that cancer outcomes could be evaluated to provide insight into disease behavior and improve cancer outcomes. It has been shown that racial disparities in cancer incidence and outcome exist for tumors at multiple anatomic sites [41].

For African Americans, there is increased incidence of SCCHN compared to people of predominantly Northern European decent (Whites). For the years 1973-1995, the incidence of oral cavity and pharyngeal cancer has increased 39.7% percent and mortality by 1.8% among African American men, while the corresponding incidence and mortality among White men has decreased 17.6% and 35.7%, respectively [42]. The trend for women is similar but less in magnitude.

Furthermore, oral cavity cancer has risen to the fourth most frequently diagnosed cancer in African American men (compared to eleventh most common for White men) with an annual incidence of 20.4/10⁶ [42]. Only prostate, lung, and colon cancer have a greater incidence is this group; of the 11 racial and ethnic groups evaluated in the SEER program, no other group has oral cancer incidence ranking in the top five. SCCHN tumors were the fourth most leading cause of cancer mortality among African American males 35-54 years old.

African Americans are diagnosed with head and neck cancer at an earlier age and more advanced stage. Previously, a review of the Tumor Registries of the East Orange, New Jersey VA Medical Center and School of Medicine and Dentistry revealed that 70% of the cases diagnosed at an age of less than 45 years were among African Americans. Sixty-one percent of this group presented with Stage III or IV; two-year survival among advanced stage disease was 23% and 40% for African Americans and Whites, respectively. Hoffman reported data from the National Cancer Database of 295,000 head and neck cancer cases for the years 1985-1994; African Americans were found to present with advanced disease (Stage III of IV) at a rate of 57.6%, while Whites only 40.3% [43]. After controlling for disease stage and epidemiologic factors, a significant outcome disparity persists.

Our recent review of our institutional experience at University of Maryland School of Medicine mirrors the racial disparity observed by others for cancer outcomes with advanced SCCHN. We evaluated 103 patients treated with a weekly Carboplatin and Taxol regimen and definitive radiation (70.2 Gy). African Americans (42%) and Whites (58%) were similar with respect to age, gender, clinical stage, tumor site, and duration of treatment. African Americans had a higher unadjusted disease recurrence rate than Whites (57% and 37% p=0.05, respectively) and failed distantly more often (27% and 12% p=0.06, respectively). When multivariable analysis was performed, African Americans independently had an increase probability for recurrence compared to Whites. Stage 1V disease and oropharyngeal tumors also were important predictors for recurrence.

We evaluated EGFR expression in a cohort of 20 African Americans. We established a reproducible immunohistochemical staining (IHC) protocol and for EGFR staining index (SI) as previously described by Ang, et al. [33d]. Previous work has shown that the overall survival and disease free survival rates of patients with high EGFR-expressing SCCHNs (>median of the mean absorbances) were highly significantly lower (P=0.0006 and P=0.0016, respectively) and the local-regional relapse rate was highly significantly higher (P=0.0031) compared with those of patients with low EGFR-expressing HNSCCs [33d]. Among the 20 African Americans tissue samples, all stained IHC positive for EGFR. Average SC staining based on tumor differentiation is as follows: Well to moderately differentiated SC=3.2; moderately differentiated SC=2.9; moderately to poorly differentiated SC=2.8; and poorly differentiated SC=2.4. The median SI for the entire cohort was 67.5.

Based upon our results with EGFR expression in non-malignant tissue from African Americans, we determined an appropriate sample size to determine if African Americans have relatively higher EGFR expression. The desired sample size was calculated to conservatively detect a 20% difference in EGFR expression SI between African Americans and Whites with a significance level α=0.05 and with power (1-β)=0.90. From our prior experiments with EGFR expression among African Americans, we noted mean SI to be 68.7 and made an assumption that SI is 20% less in Whites. A sample size to detect differences in experimental and control tumor growth with the previous mention criteria is:

n=[2*σ²*ƒ(α,β)]/[(x_{AA}−x_{W})²]

Where n is sample size for each group; x_{AA}−x_{W} are the mean EGFR expression SI for African Americans and Whites, respectively with σ representing standard deviation, (13.6). Finally, ƒ(α,β) is a function of α,β for significance level α=0.05 and power (1-β)=0.90 and has a magnitude of 10.5. Based upon these data, our treatment groups have n=21. We rounded the sample up by approximately 10% to n=25 to account for unexpected technical error or missing follow up data.

### Prophetic Example 14

### Retrospective Analysis of EGFR Expression

Using the immunohistochemical staining procedure discussed above, paraffin embedded SCCHN tissue sections are stained with an antibody against EGFR (epidermal growth factor receptor, Clone 31G7) using the VENTANA® BENCHMARK SYSTEM (Tucson, Ariz.). The stained slides are analyzed using CHROMAVISION® ACIS (Automated Cellular Imaging System, San Juan Capistrano, Calif.), which uses image capture technology to quantify EGFR staining based on the color, color purity and intensity of staining in the samples. Positive staining for EGFR is measured on a scale from 0 (no staining detected) to 4+ (maximum staining) Staining Intensity (SI) is measured on a scale of 0 (no staining detected) to 194 (maximum staining) The numerical scale used by ACIS to report SI is comparable to that used in the protocol reported by Ang, et al. [33d]. EGFR expression is determined for the cohort and over expression is based upon staining intensity levels above the median staining intensity for the cohort. [33d].

The SAS® 9.0 (Carey, N.C.) is used to perform all statistical computations. EGFR expression among African Americans and Whites is compared using Chi-square. EGFR expression measured as staining intensity is higher in tumors from African Americans relative to tumors from White subjects.

### Prophetic Example 15

### Retrospective Analysis of FcgRIIIa and FcgRIIa Polymorphisms

We determine the frequency of polymorphism for both FcgRIIIa (158 F/V) and FcgRIIa (131H/R) in tissue samples from African Americans. We purify DNA from patients' saliva, blood or from formaldehyde fixed paraffin embedded tumor samples. Allelic polymorphism analysis for both of the Fc receptors is performed as described above and shown in FIGS. 3 and 4. Comparison of FcgR polymorphisms frequencies is compared for African Americans and White subjects using Chi-square analysis. FcgRIIIa (158 F) and/or FcgRIIa (131 R) are more frequent in African Americans.

### Prophetic Example 16

### Retrospective Analysis of Recurrence in Patients Receiving C225 (Cetuximab) mAb

The cohort of patients analyzed includes patients receiving chemoradiation together with Cetuximab. We evaluate the unadjusted local-regional recurrence and disease-free rates. Additionally, we perform a multivariable regression analysis to adjust for disease and demographic variables to determine if EGFR expression, NK FcgR polymorphisms, or race/ethnicity independently predict recurrence. All statistical computations will be done with the SAS® statistical package 9.0 (Carey, N.C.). In SCCHN patients, EGFR over expression is a statistically validated independent predictor of recurrence and this correlates with differences among racial/ethnic groups. Furthermore, we verify that an ADCC mechanism plays an important role in therapeutic response, based on a correlation between clinical response to C225 therapy and FcgR affinity and polymorphisms. Monoclonal antibodies directed to EGFR, such as C225 (Cetuximab), can be optimized for Fc carbohydrate content, as described above. Fc carbohydrate is engineered to have optimal affinity to a patient's FcgR alleles to improve binding and subsequent ADCC. Alternatively, C225 carbohydrate content is selected to maximize the probability of optimal binding based on racial or ethnic FcgR allele frequencies as a surrogate for individualized genetic profiling.

### REFERENCES

All patents and publications mentioned in this specification are indicative of the level of those skilled in the art to which the invention pertains. All patents and publications herein are incorporated by reference to the same extent as if each individual publication was specifically and individually indicated as having been incorporated by reference in their entirety. All of the following references have been cited in this application:
[1] Adams, G. P., and Weiner, L. M. 2005. Monoclonal antibody therapy of cancer. Nat Biotechnol 23:1147-1157.
[2] Carter, P. 2001. Improving the efficacy of antibody-based cancer therapies. Nat Rev Cancer 1:118-129.
[2b] Shields, Robert L., et al. Lack of Fucose on Human IgG1 N-Linked Oligosaccharide Improves Binding to Human FcγRIII and Antibody-dependent Cellular Toxicity, J. Biol. Chem., Vol. 277, Issue 30, 26733-26740, Jul. 26, 2002.
[2c] Shinkawa, Toyohide, et al. The Absence of Fucose but Not the Presence of Galactose or Bisecting N-Acetylglucosamine of Human IgG1 Complex-type Oligosaccharides Shows the Critical Role of Enhancing Antibody-dependent Cellular Cytotoxicity. J. Biol. Chem., January 2003; 278: 3466-3473.
[2d] Niwa, Rinpei, et al. Defucosylated Chimeric Anti-CC Chemokine Receptor 4 IgG1 with Enhanced Antibody-Dependent Cellular Cytotoxicity Shows Potent Therapeutic Activity to T-Cell Leukemia and Lymphoma. Cancer Res. 2004 64: 2127-2133
[2e] Wu, J., et al., A novel polymorphism of FcgRIIIa (CD16a) alters receptor function and predisposes to autoimmune disease. J Clin Invest, 1997. 100(5): p. 1059-70.
[3] Weng, W. K. and R. Levy, Two immunoglobulin G fragment C receptor polymorphisms independently predict response to rituximab in patients with follicular lymphoma. J Clin Oncol, 2003. 21(21): p. 3940-7.
[4] Weng, W. K., et al., Clinical outcome of lymphoma patients after idiotype vaccination is correlated with humoral immune response and immunoglobulin G Fc receptor genotype. J Clin Oncol, 2004. 22(23): p. 4717-24.
[5] Jefferis, R. 2005. Glycosylation of recombinant antibody therapeutics. Biotechnol Prog 21:11-16.
[6] Li, H., Sethuraman, N., Stadheim, T. A., Zha, D., Prinz, B., Ballew, N., Bobrowicz, P., Choi, B. K., Cook, W. J., Cukan, M., Houston-Cummings, N. R., Davidson, R., Gong, B., Hamilton, S. R., Hoopes, J. P., Jiang, Y., Kim, N., Mansfield, R., Nett, J. H., Rios, S., Strawbridge, R., Wildt, S., and Gerngross, T. U. 2006. Optimization of humanized IgGs in glycoengineered Pichia pastoris. Nat Biotechnol 24:210-215.
[7] Lund, J., Takahashi, N., Popplewell, A., Goodall, M., Pound, J. D., Tyler, R., King, D. J., and Jefferis, R. 2000. Expression and characterization of truncated forms of humanized L243 IgG1. Architectural features can influence synthesis of its oligosaccharide chains and affect superoxide production triggered through human Fc-g receptor I. Eur J Biochem 267:7246-7257.
[8] Mimura, Y., Church, S., Ghirlando, R., Ashton, P. R., Dong, S., Goodall, M., Lund, J., and Jefferis, R. 2000. The influence of glycosylation on the thermal stability and effector function expression of human IgG1-Fc: properties of a series of truncated glycoforms. Mol Immunol 37:697-706.
[9] Krapp, S., Mimura, Y., Jefferis, R., Huber, R., and Sondermann, P. 2003. Structural analysis of human IgG-Fc glycoforms reveals a correlation between glycosylation and structural integrity. J Mol Biol 325:979-989.
[10] Mimura, Y., Sondermann, P., Ghirlando, R., Lund, J., Young, S. P., Goodall, M., and Jefferis, R. 2001. Role of oligosaccharide residues of IgG1-Fc in Fc g RIIb binding. J Biol Chem 276:45539-45547.
[11] Sondermann, P., Huber, R., Oosthuizen, V., and Jacob, U. 2000. The 3.2-A crystal structure of the human IgG1 Fc fragment-Fc gRIII complex. Nature 406:267-273.
[12] Lifely, M. R., Hale, C., Boyce, S., Keen, M. J., and Phillips, J. 1995. Glycosylation and biological activity of CAMPATH-1H expressed in different cell lines and grown under different culture conditions. Glycobiology 5:813-822.
[13] Umana, P., Jean-Mairet, J., Moudry, R., Amstutz, H., and Bailey, J. E. 1999. Engineered glycoforms of an antineuroblastoma IgG1 with optimized antibody-dependent cellular cytotoxic activity. Nat Biotechnol 17:176-180.
[14] Davies, J., Jiang, L., Pan, L. Z., LaBarre, M. J., Anderson, D., and Reff, M. 2001. Expression of GnTIII in a recombinant anti-CD20 CHO production cell line: Expression of antibodies with altered glycoforms leads to an increase in ADCC through higher affinity for FC g RIII. Biotechnol Bioeng 74:288-294.
[15] Shinkawa, T., Nakamura, K., Yamane, N., Shoji-Hosaka, E., Kanda, Y., Sakurada, M., Uchida, K., Anazawa, H., Satoh, M., Yamasaki, M., Hanai, N., and shiara, K. 2003. The absence of fucose but not the presence of galactose or bisecting N-acetylglucosamine of human IgG1 complex-type oligosaccharides shows the critical role of enhancing antibody-dependent cellular cytotoxicity. J Biol Chem 278:3466-3473.
[16] Kumpel, B. M., Rademacher, T. W., Rook, G. A., Williams, P. J., and Wilson, I. B. 1994. Galactosylation of human IgG monoclonal anti-D produced by EBV-transformed B-lymphoblastoid cell lines is dependent on culture method and affects Fc receptor-mediated functional activity. Hum Antibodies Hybridomas 5:143-151.
[17] Kumpel, B. M., Wang, Y., Griffiths, H. L., Hadley, A. G., and Rook, G. A. 1995. The biological activity of human monoclonal IgG anti-D is reduced by beta-galactosidase treatment. Hum Antibodies Hybridomas 6:82-88.
[18] Boyd, P. N., Lines, A. C., and Patel, A. K. 1995. The effect of the removal of sialic acid, galactose and total carbohydrate on the functional activity of Campath-1H. Mol Immunol 32:1311-1318.
[19] Lund, J., Takahashi, N., Pound, J. D., Goodall, M., Nakagawa, H., and Jefferis, R. 1995. Oligosaccharide-protein interactions in IgG can modulate recognition by Fc g receptors. Faseb J 9:115-119.
[20] Stanley, P. 1992. Glycosylation engineering. Glycobiology 2:99-107.
[21] Hollister, J., Grabenhorst, E., Nimtz, M., Conradt, H., and Jarvis, D. L. 2002. Engineering the protein N-glycosylation pathway in insect cells for production of biantennary, complex N-glycans. Biochemistry 41:15093-15104.
[22] Choi, B. K., Bobrowicz, P., Davidson, R. C., Hamilton, S. R., Kung, D. H., Li, H., Miele, R. G., Nett, J. H., Wildt, S., and Gerngross, T. U. 2003. Use of combinatorial genetic libraries to humanize N-linked glycosylation in the yeast Pichia pastoris. Proc Natl Acad Sci USA 100:5022-5027.
[23] Hamilton, S. R., Bobrowicz, P., Bobrowicz, B., Davidson, R. C., Li, H., Mitchell, T., Nett, J. H., Rausch, S., Stadheim, T. A., Wischnewski, H., Wildt, S., and Gerngross, T. U. 2003. Production of complex human glycoproteins in yeast. Science 301:1244-1246.
[24] Wildt, S., and Gerngross, T. U. 2005. The humanization of N-glycosylation pathways in yeast. Nat Rev Microbiol 3:119-128.
[25] Niwa, R., Sakurada, M., Kobayashi, Y., Uehara, A., Matsushima, K., Ueda, R., Nakamura, K., and shiara, K. 2005. Enhanced natural killer cell binding and activation by low-fucose IgG1 antibody results in potent antibody-dependent cellular cytotoxicity induction at lower antigen density. Clin Cancer Res 11:2327-2336.
[26] Yamane-Ohnuki, N., Kinoshita, S., Inoue-Urakubo, M., Kusunoki, M., Iida, S., Nakano, R., Wakitani, M., Niwa, R., Sakurada, M., Uchida, K., shiara, K., and Satoh, M. 2004. Establishment of FUT8 knockout Chinese hamster ovary cells: an ideal host cell line for producing completely defucosylated antibodies with enhanced antibody-dependent cellular cytotoxicity. Biotechnol Bioeng 87:614-622.
[26b] Sautes-Fridman C, C. L., Cohen-Solal J, Fridman W, Fc Gamma Receptors: A Magic Link with the Outside World. Scientific Communications, 2003: p. 148-151.
[27] Li, B., Zeng, Y., Hauser, S., Song, H., and Wang, L. X. 2005. Highly efficient endoglycosidase-catalyzed synthesis of glycopeptides using oligosaccharide oxazolines as donor substrates. J. Am. Chem. Soc. 127:9692-9693.
[28] Li, H., Li, B., Song, H., Breydo, L., Baskakov, I. V., and Wang, L. X. 2005. Chemoenzymatic Synthesis of HIV-1 V3 Glycopeptides Carrying Two N-Glycans and Effects of Glycosylation on the Peptide Domain. J Org Chem 70:9990-9996.
[29] Zeng, Y., Wang, J., Li, B., Hauser, S., Li, H., and Wang, L. X. 2006. Glycopeptide Synthesis through endo-Glycosidase-Catalyzed Oligosaccharide Transfer of Sugar Oxazolines: Probing Substrate Structural Requirement. Chem. Eur. J. 12:3355-3364.
[30] Li, B., Song, H., Hauser, S., and Wang, L. X. 2006. A Highly Efficient Chemoenzymatic Approach Toward Glycoprotein Synthesis. Org. Lett. 8:3081-3084.
[31] Watt, G. M., Lund, J., Levens, M., Kolli, V. S., Jefferis, R., and Boons, G. J. 2003. Site-specific glycosylation of an aglycosylated human IgG1-Fc antibody protein generates neoglycoproteins with enhanced function. Chem Biol 10:807-814.
[32] U.S. Pat. No. 7,138,371
[33] Bing Li, Ying Zeng, Steven Hauser, Haijing Song, and Lai-Xi Wang, Highly Efficient Endoglycosidase-Catalyzed Synthesis of Glycopeptides Using Oligosaccharide Oxazolines as Donor Substrates, J. AM. CHEM. SOC. 2005, 127, 9692-9693.
[33b] Bazley, L. A. and W. J. Gullick, The epidermal growth factor receptor family. Endocr Relat Cancer, 2005. 12 Suppl 1: p. S17-27.
[33c] Choong, N. W. and E. E. Cohen, Epidermal growth factor receptor directed therapy in head and neck cancer. Crit Rev Oncol Hematol, 2006. 57(1): p. 25-43.
[33d] Ang, K. K., et al., Impact of epidermal growth factor receptor expression on survival and pattern of relapse in patients with advanced head and neck carcinoma. Cancer Res, 2002. 62(24): p. 7350-6.
[33e] Bonner, J. A., et al., Radiotherapy plus cetuximab for squamous-cell carcinoma of the head and neck. N Engl J Med, 2006. 354(6): p. 567-78.
[34] Tournoy, K., S. Depraetere, P. Meuleman, G. Leroux-Roels, R. Pauwels. 1998. Murine interleukin 2 receptor chain blockade improves human leukocyte engraftment in severe combined immunodeficient (SCID) mice. Eur. J. Immunol 28:3221.
[35] Shibata, S., T. Asano, A. Noguchi, H. Kimura, A. Ogura, M. Naiki, K. Doi. 1998. Enhanced engraftment of human peripheral blood lymphocytes into anti-murine interferon-monoclonal antibody-treated C. B.-17-scid mice. Cell. Immunol. 183:60.
[36] Mueller B M, Romerdahl C A, Trent J M, Reisfeld R A. Suppression of spontaneous melanoma metastasis in scid mice with an antibody to the epidermal growth factor receptor. Cancer Res. 1991 Apr. 15; 51(8):2193-8.
[37] Goldstein N I, Prewett M, Zuklys K, Rockwell P, Mendelsohn J. Biological efficacy of a chimeric antibody to the epidermal growth factor receptor in a human tumor xenograft model. Clin Cancer Res. 1995 November; 1(11):1311-8.
[38] Naramura M, Gillies S D, Mendelsohn J, Reisfeld R A, Mueller B M. Therapeutic potential of chimeric and murine anti-(epidermal growth factor receptor) antibodies in a metastasis model for human melanoma. Cancer Immunol Immunother. 1993 October; 37(5):343-9.
[39] Naramura M, Gillies S D, Mendelsohn J, Reisfeld R A, Mueller B M. Therapeutic potential of chimeric and murine anti-(epidermal growth factor receptor) antibodies in a metastasis model for human melanoma. Cancer Immunol Immunother. 1993 October; 37(5):343-9.
[40] Hodoniczky J, Zheng Y Z, James D C. Control of recombinant monoclonal antibody effector functions by Fc N-glycan remodeling in vitro. Biotechnol Prog. 2005 November-December; 21(6):1644-52.
[41] Ward, E., et al., Cancer disparities by race/ethnicity and socioeconomic status. CA Cancer J Clin, 2004. 54(2): p. 78-93.
[42] NationalCancerinstitute, SEER Cancer Statistics Review 1973-1995. [Web page], 1998.
[43] Hoffman, H. T., et al., The National Cancer Data Base report on cancer of the head and neck. Arch Otolaryngol Head Neck Surg, 1998. 124(9): p. 951-62.
[44] Nakayama, K., T. Nagasu, Y. Shimma, J. Kuromitsu, and Y. Jigami. 1992. OCH1 encodes a novel membrane bound mannosyltransferase: outer chain elongation of asparagine-linked oligosaccharides. EMBO J. 11:2511-2519.
Jemal, A., et al., Cancer statistics, 2004. CA Cancer J Clin, 2004. 54(1): p. 8-29.
Shuch, B., et al., Racial disparity of epidermal growth factor receptor expression in prostate cancer. J Clin Oncol, 2004. 22(23): p. 4725-9.
Coura, R., et al., An alternative protocol for DNA extraction from formalin fixed and paraffin wax embedded tissue. J Clin Pathol, 2005. 58(8): p. 894-5.
Yang, S. H., et al., Mutations in the tyrosine kinase domain of the epidermal growth factor receptor in non-small cell lung cancer. Clin Cancer Res, 2005. 11(6): p. 2106-10.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of producing an antibody having a desired glycosylation state comprising the steps of
a) removing one or more sugars,
b) chemically synthesizing a sugar, and
c) enzymatically attaching the chemically synthesized sugar to (i) the antibody or (ii) a sugar attached to the antibody.

2. The method of claim 1, wherein the chemically synthesized sugar comprises an oxazoline ring.

3. The method of claim 1, wherein the enzyme is an endoglycosidase and the enzymatic attachment comprises a transglycosylation.

4. The method of claim 1, wherein the sugar removed is an asparagine linked sugar, the polypeptide retains an N-acetylglucosamine at the asparagine after step a) and the enzymatic attachment is to the N-acetylglucosamine.

5. The method of claim 1, wherein the antibody is a monoclonal antibody and the method results in substantially pure monoclonal antibody.

6. The method of claim 1, wherein the chemically synthesized sugar results in a non-natural carbohydrate structure after step c).

7. The method of claim 5, wherein the substantially pure monoclonal antibody comprises a glycosylation state capable of modulating a biological activity.

8. The method of claim 7, wherein the biological activity is (i) a binding affinity for an Fcg Receptor or (ii) antibody-dependent cell-mediated cytotoxicity.

9. The method of claim 5, wherein the monoclonal antibody comprises cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101, volociximab, Anti-CD80 mAb, Anti-CD23 mAb, CAT-3888, CDP-791, eraptuzumab, MDX-010, MDX-060, MDX-070, matuzumab, CP-675,206, CAL, SGN-30, zanolimumab, adecatumumab, oregovomab, nimotuzumab, ABT-874, denosumab, AM 108, AMG 714, fontolizumab, daclizumab, golimumab, CNTO 1275, ocrelizumab, HuMax-CD20, belimumab, epratuzumab, MLN1202, visilizumab, tocilizumab, ocrerlizumab, certolizumab pegol, eculizumab, pexelizumab, abciximab, ranibizimumab, mepolizumab, TNX-355, or MYO-029.

10. An antibody composition comprising antibodies having a substantially pure glycosylation state.

11. The antibody composition of claim 10, wherein the glycosylation state comprises at least four sugars.

12. The antibody composition of claim 10, wherein the antibody is a monoclonal antibody.

13. The antibody composition of claim 12, wherein the monoclonal antibody comprises cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101, volociximab, Anti-CD80 mAb, Anti-CD23 mAb, CAT-3888, CDP-791, eraptuzumab, MDX-010, MDX-060, MDX-070, matuzumab, CP-675,206, CAL, SGN-30, zanolimumab, adecatumumab, oregovomab, nimotuzumab, ABT-874, denosumab, AM 108, AMG 714, fontolizumab, daclizumab, golimumab, CNTO 1275, ocrelizumab, HuMax-CD20, belimumab, epratuzumab, MLN1202, visilizumab, tocilizumab, ocrerlizumab, certolizumab pegol, eculizumab, pexelizumab, abciximab, ranibizimumab, mepolizumab, TNX-355, or MYO-029.

14. A method of evaluating a biological activity of a glycopolypeptide comprising the steps of
a) producing a substantially pure population of glycopolypeptides having a selected glycosylation state, and
b) measuring the biological activity of the glycopolypeptide.

15. The method of claim 14, wherein the glycopolypeptide is an antibody and the biological activity is (i) a binding affinity for an Fcg Receptor or (ii) antibody-dependent cell-mediated cytotoxicity.

16. The method of claim 15, wherein the antibody comprises a monoclonal antibody.

17. The method of claim 15, wherein the biological activity is antibody-dependent cell-mediated cytotoxicity in vivo.

18. The method of claim 16, wherein the monoclonal antibody comprises cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101, volociximab, Anti-CD80 mAb, Anti-CD23 mAb, CAT-3888, CDP-791, eraptuzumab, MDX-010, MDX-060, MDX-070, matuzumab, CP-675,206, CAL, SGN-30, zanolimumab, adecatumumab, oregovomab, nimotuzumab, ABT-874, denosumab, AM 108, AMG 714, fontolizumab, daclizumab, golimumab, CNTO 1275, ocrelizumab, HuMax-CD20, belimumab, epratuzumab, MLN1202, visilizumab, tocilizumab, ocrerlizumab, certolizumab pegol, eculizumab, pexelizumab, abciximab, ranibizimumab, mepolizumab, TNX-355, or MYO-029.

19. A method of improving the outcome of an antibody based therapy comprising the steps of
a) determining for a subject an Fcg Receptor allele present in a subject, and
b) treating the subject with a monoclonal antibody comprising a substantially pure glycosylation state selected for (i) increased binding affinity to the Fcg Receptor allele present in the subject or (ii) increased antibody-dependent cell-mediated cytotoxicity.

20. The method of claim 19, wherein the Fcg Receptor allele is an FcgIIIa Receptor allele for amino acid 158 or an FcgIIa Receptor allele for amino acid 131.

21. A method of selecting the glycosylation state for a monoclonal antibody comprising the steps of
a) determining a Fcg Receptor allele on an immune cell, and
b) selecting a glycosylation state which modulates, relative to a source monoclonal antibody having a heterogeneous glycosylation state,
i) Antibody Dependent Cell Cytotoxicity,
ii) Complement Dependent Cytotoxicity,
iii) an Fc g receptor binding affinity, or
iv) a monoclonal antibody induced cell signaling event.

22. A method of creating a bioequivalent of a monoclonal antibody comprising the steps of
a) determining a glycosylation state for a pre-existing monoclonal antibody, and
b) using the method of claim 1 to produce a monoclonal antibody having substantially the same glycosylation state as the pre-existing monoclonal antibody.

23. A method of selecting for clinical development a glycoform of a monoclonal antibody for use in a population having an Fcg receptor allele comprising the steps of
a) testing a glycoform of a monoclonal antibody for biological activity against the Fcg Receptor alleles present in the population, and
b) selecting for clinical development the monoclonal antibody glycoform capable of (i) increased binding affinity to the Fcg Receptor allele present in the population or (ii) increased antibody-dependent cell-mediated cytotoxicity.

24. The method of claim 23, wherein the Fcg Receptor allele is an FcgIIIa Receptor allele for amino acid 158 or an FcgIIa Receptor allele for amino acid 131.

25. A method of creating a substantially pure glycoform of a pre-existing monoclonal antibody having a heterogeneous glycosylation state comprising the steps of
a) using the method of claim 1 to create two or more of the glycoforms present in the pre-existing monoclonal antibody,
b) testing the two or more glycoforms for a biological activity or a toxicity to determine a preferred glycoform of the pre-existing monoclonal antibody having a higher biological activity or a lower toxicity, and
using the method of claim 1 to produce a monoclonal antibody glycoform having a substantially pure preferred glycosylation state identified in step b) as having a higher biological activity or a lower toxicity.


SEQUENCE LISTING (4 sequences)

SEQ ID NO: 1 - DNA, 30 bp, Artificial Sequence
  chemically synthesized PCR primer
ataaggtcacatatttacagaatggccaag

SEQ ID NO: 2 - DNA, 30 bp, Artificial Sequence
  chemically synthesized PCR primer
cagtctctgaagacacatttttactccgta

SEQ ID NO: 3 - DNA, 22 bp, Artificial Sequence
  chemically synthesized PCR primer
ggaaaatcccagaaattctcgc

SEQ ID NO: 4 - DNA, 27 bp, Artificial Sequence
  chemically synthesized PCR primer
caacagcctgactacctattacgcggg